(19) United States
(12) Patent Application Publication
(10) Pub. No.: US 2022/0233235 A1
(43) Pub. Date: Jul. 28, 2022

(54) Ablation Catheter for Pulsed-Field Ablation and Method for Electrode Position Assessment for Such Catheter

(71) Applicant: CRC EP, Inc., Lake Oswego, OR (US)

(72) Inventors: Henning Ebert, Berlin (DE); Steffen Holzinger, Berlin (DE); Dorin Panescu, San Jose, CA (US)

(73) Assignee: CRC EP, Inc., Lake Oswego, OR (US)

(21) Appl. No.: 17/563,620

(22) Filed: Dec. 28, 2021

Related U.S. Application Data
(60) Provisional application No. 63/140,390, filed on Jan. 22, 2021.
(60) Provisional application No. 63/270,666, filed on Oct. 22, 2021.

(30) Foreign Application Priority Data
May 5, 2021 (EP) 21172336.6

(51) Int. Cl. A61B 18/14

(52) U.S. Cl. CPC A61B 18/1492; A61B 2018/00577

(57) ABSTRACT

A system for treatment of patient tissue by delivery of high-voltage pulses comprising an ablation catheter, a measurement unit and an electronic control unit (ECU). The measurement unit is configured to perform measurements using an energy source, whereby the impedance and/or current measurement values are determined as response to an alternating voltage and/or at least one voltage pulse. The ECU is configured to receive and analyze said measurement values provided by the measurement unit and determine arcing risk (AR) indexes for said electrode pairs and/or a contact uniformity (CU) value based on said impedance measurement values and/or impedances for said electrodes and/or an impedance uniformity (IU) value based on said current measurement values.

### CROSS REFERENCE TO RELATED APPLICATIONS

This patent application claims the benefit of and priority to co-pending U.S. Provisional Patent Application No. 63/270,666, filed Oct. 22, 2021, and European Patent Application No. EP 21172336.6, filed May 5, 2021, and U.S. Provisional Patent Application No. 63/140,390, filed Jan. 22, 2021, which are hereby incorporated by reference in their entireties.

### TECHNICAL FIELD

The present invention relates to embodiments of a system comprising an ablation catheter suitable for pulsed-field ablation (PFA), a method for assessment of positions and/or configuration of electrodes of such ablation catheter, a respective computer program product and a respective computer readable data carrier.

### BACKGROUND

In particular, the present invention relates to embodiments of a system comprising a PFA catheter, a measurement unit and an electronic control unit, whereby the system may be used for safely performing cardiac ablation procedures, such as, but not limited to, pulmonary vein isolation (PVI), persistent atrial fibrillation ablation, ventricular tachycardiac ablation. The catheter comprises multiple electrodes and delivers pulsed-field energy to achieve irreversible electroporation of cardiac tissue.

It is known to use ablation catheters for PVI procedures in the therapy of atrial fibrillation (AF) patients. In such procedures, the pulmonary veins (PV) are electrically isolated from the left atrium by creating contiguous circumferential ablation lesions around the pulmonary vein ostium (PVO) or around their antrum. Thus, irregular atrial contractions can be avoided by hindering undesired perturbing electrical signals generated within the PV from propagating into the left atrium. Ablation catheters may be used to deliver therapy to other tissues, such as, but not limited to: ventricles, right atrium, the body of the left atrium, etc. Additionally, other organs may be treated via use of catheters: lungs, liver, kidneys, etc.

Several types of ablation catheters are available including single point tip electrode catheters, circular multi-electrode loop catheters, and balloon-based ablation catheters using different energy sources. They all lack the ability of producing the required ablations, which safely electrically isolate the arrhythmogenic triggers from the rest of the heart chamber, in a ‘one-shot’ modality, without further repositioning, rotating or moving of the catheter. It is one goal of ablation catheter development to provide catheters and systems which safely achieve a ‘moat’ of electrical isolation in one shot. The concept of a moat of electrical isolation is defined as region of cardiac tissue that surrounds the arrhythmogenic trigger and prevents its propagation to the rest of the heart chamber. For example, without limitation, referring to situations when the arrhythmogenic triggers reside inside a pulmonary vein, an ablation region which completely renders non-viable the tissue located at the vein ostium or antrum, securing transmurality, would represent said moat of electrical isolation. Pulsed-field ablation (PFA), if designed appropriately, may have the advantage of creating these conduction block/electrical isolation moats in one shot, safely without or with minimal collateral tissue damage.

An ablation catheter that is particularly well suited for PFA treatment of a patient's tissue, for example for a PVI procedure at a patient's heart tissue or vein tissue, comprises an elongated catheter shaft and an ablation portion being arranged at a distal end of the catheter shaft with a plurality of electrodes accommodated along the ablation portion, wherein the ablation portion comprises at least two loop sections forming a three-dimensional spiral or similar flexible structures that allow one electrodes to move relative to another. PFA uses high-intensity electrical fields. Under some circumstances of the treatment the distance of two electrodes may become so small that an electromagnetic field intensity is sufficiently high to ionize the medium between these electrodes. In such case, arcing develops, in particular, if bipolar PFA is used. This means that for catheters with open loops or flexible splines, some electrode pairs can approach such that the risk for arcing is increased. Arcing presents an increased level of danger to patients, as it results in unintended tissue damage.

Furthermore, the high temperatures of arcs may melt catheter materials, leaving foreign particles in the patient's blood stream.

Accordingly, determining the position and/or configuration of the electrodes is essential for catheters operated with bipolar PFA and where electrode distances between each other can change due to manipulation (especially if electrodes on different polarities come close).

Another important parameter for the success of the PFA treatment of a patient is an information about good or poor positions of the electrodes with regard to their contact with the patient tissue and therefore the quality of catheter position for the treatment. The fact that one or some ablation electrodes of the ablation portion do not have sufficient contact to the targeted tissue may impede the creation of the above-mentioned moat of electrical isolation in one shot.

International Publication No. WO 2018/102376 discloses a method of detecting arcing in an electroporation system including a direct current (DC) energy source, a return electrode connected to the DC energy source and a catheter connected to the DC energy source. The known method includes monitoring a system impedance with the return electrode positioned near the target location and the catheter electrode positioned within the body, detecting a positive deflection in the system impedance, the positive deflection indicative of arcing, and generating an alert, based on the detection, the alert indicating that arcing has occurred. The known method does only derive some information about arcing for unipolar ablation though. For bipolar PFA which is the preferred method to produce a moat of electrical isolation in one shot the known method does not give any meaningful values and adding external impedances does not necessarily prohibit arcing.

Accordingly, it is an objective of the present invention to provide reliable information about arcing risk and/or electrode contact to the operating health care professional (HCP) with regard to a bipolar PFA system in an easily understandable, reliable and time-effective way.

The present disclosure is directed toward overcoming one or more of the above mentioned problems, though not necessarily limited to embodiments that do.

### SUMMARY

At least the above problem is solved by a system having the features of claim 1, a method for assessment of positions and/or configuration of a plurality of electrodes having the features of claim 13 a computer program product with the features of claim 25 and a computer readable data carrier with the features of claim 26.

In particular, at least the above problem is solved by a system for treatment of patient tissue by delivery of high-voltage pulses comprising an ablation catheter, a measurement unit and an electronic control unit, whereby the catheter comprises a catheter shaft and an ablation portion being arranged at a distal end of the catheter shaft with a plurality of electrodes accommodated along the ablation portion, wherein each of the plurality of electrodes is electrically connected to a measurement unit through the catheter shaft, wherein the measurement unit is configured to perform measurements using an energy source thereby determining measurement values, in particular bipolar impedance measurement values of electrode pairs of a subgroup of the plurality of electrodes and/or quasi-unipolar impedance measurement values of a subgroup of the plurality of electrodes and/or current measurement values of a subgroup of the plurality of electrodes, wherein said subgroup is formed by all or a part of the plurality of electrodes, respectively, whereby the impedance and/or current measurement values may be determined as response to an alternating voltage and/or at least one voltage pulse, wherein the electronic control unit (ECU) may be arranged proximal to or at the proximal end of the catheter, wherein the measurement unit may be connected to or integrated within the ECU, wherein the ECU is configured to receive and analyze said measurement values provided by the measurement unit and determine arcing risk (AR) and/or a contact uniformity (CU) and/or an impedance uniformity (IU) indexes based on measurement values.

The arcing risk and/or the contact uniformity indexes may be based on the impedance measurement values and/or impedance for said electrodes. The impedance uniformity indexes may be based on the current measurements.

The above ablation catheter includes hardware and a respective algorithm to reliably indicate the risk for arcing and contact uniformity of electrodes. CU is important as it provides an immediate understanding to operating HCPs about the tissue contact uniformity over all active electrodes.

Within the frame of this application, the phrase “subgroup of electrodes” is understood as a pre-defined group of electrodes of the plurality of electrodes of the ablation portion of the ablation catheter which may be formed by all electrodes of the ablation portion or a real part of the electrodes of the ablation portion. For example, the ablation portion may comprise ablation electrodes and mapping electrodes as described below. In this example, the subgroup of electrodes may contain the ablation electrodes, only, but not the mapping electrodes (i.e. electrodes exclusively used for mapping).

In accordance with an embodiment, the system is configured for delivering pulsed-field ablating (PFA) energy to the patient's tissue by a health care practitioner (HCP), for example to the atrial or ventricular tissue of the patient's heart, via electrodes (also referred to as ablation electrodes) located along the ablation portion at the distal end of the ablation catheter. In other words, the system may be configured for carrying out PFA. In particular, the ablation catheter may be used to provide cardiac catheter ablation to treat a variety of cardiac arrhythmias including AF. For example, the system may comprise a multi-channel PF energy generator and the ablation catheter may be configured for being connected to a multi-channel PF energy generator which is configured for delivering PF energy. The waveform of said PF energy generator is conceived so that it, in conjunction with catheter loop or spiral design, achieves intended therapeutic effect while minimizing or reducing chances of ionization and the intended impedance and current measurements as indicated above. The inventive catheter may also be used for different type of tissue, for example veins, lungs, liver, kidneys. It may be used for pulmonary vein isolation (PVI), persistent atrial fibrillation ablation, ventricular tachycardiac ablation and other ablation procedures.

The inventive ablation catheter using PFA is intended to render tissues non-viable by irreversible electroporation (IRE). During IRE the electric field provided by the electrodes accommodated at the neighboring loop sections creates pores in cardiac cell membranes.

When the number of pores and their sizes are sufficiently great IRE occurs and the cell programs itself to die. For that neighboring loop sections of the ablation portion form a so-called ablation area.

The system comprises a measurement unit and an electronic control unit (ECU). The system may further comprise a multi-channel PF energy generator (further referred as PF generator) as an energy source. The measurement unit and the ECU may be integrated in the PF generator. The electronic control unit (ECU) may also be configured for controlling ablation procedure, in particular the PF generator, and receiving, processing and analyzing measurement values. The ECU comprises a microprocessor, computer or the like and is regarded as a functional unit of the system that interprets and executes instructions comprising an instruction control unit and an arithmetic and logic unit.

The catheter shaft may comprise a handle at its proximal end. Each electrode of the plurality of electrodes at the ablation portion is electrically connected via one electrical conductor to the PF generator provided at the proximal end of the catheter shaft. In an alternative embodiment, the measurement unit and/or the ECU may be at least partially integrated in the handle.

The PF generator, ECU and/or the measurement unit may be connected to or may comprise a memory module for storing data, e.g. measurement values or determined data calculated by the ECU from these measurement values. The memory module of the PF generator, the ECU and/or the measurement unit may include any volatile, non-volatile, magnetic, electrical media, or otherwise such as a random access memory (RAM), read-only memory (ROM), non-volatile RAM (NVRAM), electrically-erasable programmable ROM (EEPROM), flash memory, or any other memory storage type. The ECU may further be connected to a (graphical) user interface (GUI), e.g. for the HCP, in order to receive data input and/or display the determined AR indexes, CU value, impedance values and/or IU value.

In another embodiment, there are two electrical conductors provided at the proximal end and the middle section of the catheter shaft. At the proximal end, the first electrical conductor is connected to the first group of electrodes and the second electrical conductor is connected to the second group of electrodes in order to reduce the diameter of the catheter shaft. One electrode consists of electrically conducting material, for example, at least one of gold and a platinum/iridium alloy and/or may have a length along the respective ablation portion section of 1 mm to 10 mm, preferably 3 mm to 5 mm. The catheter shaft size may be compatible with a 7 F to 14 F ID sheath, preferable with an 8.5 F ID sheath. The width between adjoining electrodes along the respective loop section may be chosen between 1 mm and 10 mm, preferably 3-6 mm, in order to provide a contiguous ablated area at the patient's tissue.

In one embodiment, the length of the ablation electrodes may be in the range 3-5 mm. In one embodiment, the ablation electrodes may be sleeve-shaped or tubular. For example, a diameter of such a sleeve-shaped or tubular ablation electrode may be in the range of 2-2.5 mm. Further, as mentioned above, a length of the sleeve-shaped or tubular ablation electrode may be in the range of 1-10 mm, for example 3-5 mm. Alternatively, a split electrode design may be used. In this embodiment, two electrodes in form of half-shells separated by a gap are arranged at the inner side (facing the body lumen) and the outer side (facing the tissue) of the catheter. The gap may be 0.2-1 mm wide, preferably 0.5 mm wide. Alternatively, electrodes may be solid but coated with insulating material on the inner side facing blood (the body lumen). Parylene, Polyimide or Teflon are examples of a suitable coating. The coating material should be an electrical insulator with high dielectric strength, in excess of 200 kV/mm.

Each of the electrodes is electrically connected to the electronic control unit (ECU), wherein the connection may be provided via the PF generator to pair each two of at least two electrodes of the subgroup of electrodes in a pre-defined manner in order to operate the electrodes in a bipolar arrangement. If there are more than two electrodes, for example 16 electrodes, e.g. each two electrodes which are accommodated adjacent along the ablation portion may be paired (mode along the ablation portion) or each two electrodes which are accommodated adjacent across two neighboring loop sections of the ablation portion (see description of loop structure below, mode across loop sections) may be paired to be operated in a bipolar arrangement. Accordingly, 8 pairs may be formed from 16 electrodes in both modes. The pairing may be switched between the two modes. Further, the pairing may be switched to another pair of electrodes, for example along the loop sections. For pairing, the electrodes may be connected to a switch unit, wherein the switch unit is connected and controlled by the ECU. The ECU may further be adapted to switch into the below-mentioned ablation mode and mapping mode for each electrode, respectively. The switch unit realizes the pairing along the loop sections and, if applicable, the switching between the modes according to the control signals of the ECU.

In one embodiment, the measurement unit may be separate from or integrated within the ECU, wherein the measurement unit is configured to provide an activation signal in form of an AC voltage signal, AC current signal or at least one voltage pulse. In the case that the measurement unit is separate from the ECU, the measurement unit is electrically connected to the ECU.

In one embodiment, the measurement unit is configured to determine at least one current measurement value for each of the subgroup of the plurality of electrodes by measuring the respective current value of one or several of rectangular, sinusoidal, tooth or similar shaped voltage pulses, wherein one impedance value is determined from said determined current measurement values for each of the subgroup of electrodes. In this embodiment, the current measurement value is determined by a quasi-unipolar arrangement, wherein one of the electrodes of the ablation portion forms the reference electrode. In other words, the impedance value for an electrode is determined by using this electrode as reference electrode and measuring the current values in response to the voltage pulse at least one electrode of the subgroup of electrodes, in particular the current values of all or selected electrodes of different polarity of the subgroup compared to the reference electrode. For each of the electrodes the peak current is determined, wherein the peak voltage may be chosen between 1V and 1 kV, in particular between 10V and 700V, in particular between 100V and 500V. Each pulse comprises a positive and a negative half-wave having a rectangular, sinusoidal, tooth or similar shaped voltage pulse. The ECU analyzes the measurement values received from the measurement unit and determines from the peak voltage and the measured peak current value the impedance value for each electrode separately, wherein a mean value is determined for each electrode if the peak current is determined from more than one voltage pulse for each electrode. The frequency of the voltage pulse is, for example, 500 kHz. The determined impedances for each electrode may be presented to the HCP, for example, by means of a bar diagram, wherein the height of the bar represents the impedance value of the respective electrode. In one embodiment, the impedances for different positions of the ablation catheter with regard to the tissue may be presented for each electrode side by side. Further, a mean impedance value may be determined from all electrodes of the subgroup or a group of the subgroup, for example the proximal group and distal group. If the impedance value differs from the respective mean impedance value by a pre-defined percentage, the respective bar may be colored or otherwise highlighted thereby indicating to the HCP that the respective electrode is short circuited or malfunctional.

In one embodiment, the ECU is configured to determine an impedance uniformity (IU) value of the electrodes, wherein IU*=1−½*(σ({Zₙ})/μ({Zₙ})), wherein Zₙ is the impedance of the electrode n and σ({Zₙ}) is the standard deviation of the impedance values of all electrodes of the subgroup and μ({Zₙ}) is the mean value of the impedance values of all electrodes of the subgroup. In an alternative embodiment, the mean value and the standard deviation of two groups of the subgroup of electrodes are calculated separately, such that

$$IU = 1 - \frac{1}{2} ( \frac{σ ( \{ {Z}_{d} \} )}{μ ( \{ {Z}_{d} \} )} + \frac{σ ( \{ {Z}_{p} \} )}{μ ( \{ {Z}_{p} \} )} ) ,$$

wherein σ({Z_{d,p}}) is the standard deviation and μ({Z_{d,p}}) the mean value of the respective of the impedances of the electrodes of the respective group d/p, determined by the current measurement for each electrode as indicated above. The impedance values used for IU/IU*-determination are the impedances determined by current measurements as indicated above. The IU/IU* value provides a measure that indicates whether all catheter electrodes have equal contact with their surroundings or not, for example with the targeted tissue. To account for design differences, it can be evaluated by the range of impedance values for different groups of electrodes of the subgroups, for example, the distal group (d) and the proximal group (p). An IU/IU* value close to 1 indicates that all electrodes have identical contact. If the IU/IU* is low, for example, between 0.8 and 0.9, the contact uniformity is regarded mediocre, whereas IU/IU* below 0.8 is identified as bad contact uniformity. In this case, the HCP needs to change the position of the ablation catheter, in particular the position of the ablation portion with regard to the targeted tissue in order to increase contact uniformity. Such situation must be recognized prior high energy delivery during the patient's treatment.

The impedance uniformity explained above has the same function as the CU. Both parameters are important as they provide an immediate understanding to operating HCPs about the tissue contact uniformity over all ablation electrodes.

The AR index indicates the risk for arcing for a particular electrode pair. This parameter is essential for ablation catheters which operate at bipolar PFA and where electrode distances among each other can change due to manipulation, in particular where electrodes of different polarity come close. The AR index predicts opposing electrodes well and indicates their proximity. Furthermore, a strong correlation between the AR index and an actual arcing threshold for a given PF energy is observed. Additionally, the AR index may be displayed to the HCP prior treatment in an easy way. The AR index provides a more robust approach compared to a simple impedance measurement and avoids cross-sensitivity.

In one embodiment, the ECU is configured to determine the AR index for a particular electrode pair x,y from the bipolar impedance measurement values of the particular electrode pair x,y from the subgroup of electrodes scaled by the minimum of bipolar impedance measurement values of the respective electrodes with their adjoining electrodes of the subgroup. The bipolar impedance of a particular electrode pair x,y may be measured by applying an AC voltage for example at 500 kHz between the electrodes x,y. In this embodiment adjoining electrodes along the ablation portion of the subgroup have consecutive numbering. This means that adjoining electrodes identifiable with a consecutive numbering show their risk for arcing if one uses the AR index, wherein the AR index may be calculated using the bipolar impedance measurement values Z_{x,y} for the respective pair of electrodes x,y. For a non-uniform contact of all electrodes (Z_{x,x+1} of the electrodes can be quite different, thus this should be considered as the general case), each non-adjoining measurement will get an RV-value, where x and y are the index of the electrodes, so that

$${AR}_{x , y} = 1 - \frac{{Z}_{x , y}}{min ( {Z}_{x - 1 , x} , {Z}_{x , x + 1} , {Z}_{y - 1 , y} , {Z}_{y , y + 1} )} .$$

The AR index of a particular electrode pair x, y of the subgroup is defined between 0 (low AR risk) and 1 (high AR risk). If the calculated number becomes negative, the AR_{x,y} is set to zero. Z_{x-1,x} Z_{x,x+1} refer to bipolar impedances of the electrode x and its adjoining electrodes, whereas Z_{y-1,y} Z_{y,y+1} refer to bipolar impedances of the electrode y and its adjoining electrodes. The expression min ( . . . ) defines the minimum of the impedance values indicated in parenthesis. It was shown in experiments that an AR index greater than 0.25 or, in another embodiment, greater than 0.15, causes arcing for the intended PF energy, wherein the AR index not only considers electrodes located in close and direct proximity but also electrodes which are close at their edges as these configuration may have an arcing threshold (voltage at which arcing occurs) that is lower than the maximum amplitude used for treatment for the particular ablation catheter.

In one embodiment, an AR index threshold, for example 0.15, may be defined which denotes a threshold from which arcing is observed for a particular catheter type and pulse protocol. I.e. if an AR index equal to or greater than the AR index threshold is observed, arcing is most likely noticed for the respective electrode pair. The threshold value may be defined such that it always addresses edge-edge positions. With staggered electrodes the value remains the same, but the likelihood of getting high AR values will be dramatically minimized. Staggered electrodes should be understood as an arrangement of the electrodes at the distal end of the ablation catheter whereby electrodes at the same polarity are geometrically closest. In that case the threshold may be determined more conservatively and uses even lower numbers. It is important to note that this relationship is only valid for the chosen pulse protocol as this influences the arcing threshold. Moreover, modifications of the catheter design, such as electrode length and spacing, also influence the relationship of arcing threshold and AR index. In the case that electrode lengths are not constant (e.g. use of 4 mm and 3 mm electrodes), one may add weights to the algorithm to take the different surface areas (which change the magnitude of Z) into account.

The AR index of the electrodes of the pairs x,y of the subgroup may be displayed to the HCP by means of a two-dimensional matrix (rows and lines referring to the electrode number and each intersection referring to the respective electrode pair x,y) highlighting the adjoining and/or opposing electrodes (e.g. electrodes of opposing loops of the ablation section), for example. Alternatively, the electrode pairs exhibiting a higher AR index may be highlighted at a respective visual representation of the ablation section and its electrodes located along this ablation section. The HCP may easily recognize from such visualization whether a repositioning of the catheter with regard to the patient's tissue is necessary.

In one embodiment, the ECU is configured to determine an overall risk for arcing for all electrodes of the subgroup ARₘₐₓ based on a maximum of the AR index of all electrode pairs of the subgroup, for example determined as indicated above. The overall risk for arcing refers to a particular position of the ablation catheter with regard to the patient's tissue to be treated.

ARₘₐₓ=max(AR_{x,y})

The CU value is a parameter that indicates the quality of contact of all electrodes of the pre-defined subgroup, wherein the CU value refers to a specific position of the ablation catheter with regard to the patient's tissue to be treated. For a reliable ablation treatment result, the contact of the electrodes of the subgroup should be uniform over the whole subgroup.

In one embodiment, the ECU is configured to determine the CU value for the subgroup of electrodes based on the standard deviation of the bipolar impedance measurement values of the pairs of adjoining electrodes of said subgroup or based on the minimum and the maximum of the bipolar impedance measurement values of the pairs of adjoining electrodes of said subgroup. The bipolar impedance of a particular electrode pair x,y may be measured by applying an AC voltage for example at 500 kHz between the electrodes x,y. In one embodiment, the standard deviation of the bipolar impedance measurement values is compared with the mean value of these measurement values. For example,

$$CU = 1 - \frac{σ ( \{ {Z}_{n , n + 1} \} )}{μ ( \{ {Z}_{n , n + 1} \} )} ,$$

wherein μ({Z_{n,m+1}}) is the mean value of two adjoining electrodes of the subgroup and σ({Z_{n,n+1}}) is the standard deviation of these electrodes. It was observed that good contact uniformity is realized if a CU value of about 1 (i.e. small standard deviation) is determined and a very heterogenous contact is detected if the CU value is close to zero. It is noted, that a good contact uniformity exists, too, if there are all electrodes of the subgroup without contact, e.g. floating in the blood pool. For example, a very uniform contact is given by CU values greater than 0.95. Mediocre CU is observed at a CU value of less than 0.90.

In an alternative embodiment, the CU value may be determined by the ECU using following calculation rule:

$${CU}^{′} = 1 - \sqrt{\frac{σ ( \{ {Z}_{n , n + 1} \} )}{μ ( \{ {Z}_{n , n + 1} \} )}}$$

This embodiment is basically identical to the above calculation rule for CU but provides a more “spread out” of the CU data. A mediocre CU′ is observed for CU′=0.72 and a bad CU for a CU′ value of 0.67 and less.

In an alternative embodiment, the CU value may be determined by the ECU using the following calculation rule:

$${CU}^{″} = min ( 1 - \frac{max ( \{ {Z}_{n , n + 1} \} ) - μ ( \{ {Z}_{n , n + 1} \} )}{μ ( \{ {Z}_{n , n + 1} \} )} , 1 - \frac{μ ( \{ {Z}_{n , n + 1} \} ) - min ( \{ {Z}_{n , n + 1} \} )}{μ ( \{ {Z}_{n , n + 1} \} )} ) ,$$

wherein min and max refer to the respective minimum and maximum value, respectively. This embodiment puts stronger emphasis on outliers as it compares the maximum and minimum values of Z to the average. It was observed that a CU″ value of 0.83 refers to a mediocre CU, whereas a CU″ value of 0.79 or less has a bad CU.

In an alternative embodiment, the CU value may be determined by the ECU using the following calculation rule:

$${CU}^{′′′} = 1 - \frac{max ( \{ {Z}_{n , n + 1} \} ) - min ( \{ {Z}_{n , n + 1} \} )}{μ ( \{ {Z}_{n , n + 1} \} )} .$$

This embodiment has the same tendency as the above calculation rule for CU″ and puts even more emphasis on outliers. It was observed that a CU′″ value of 0.68 refers to a mediocre CU, whereas a CU′″ value of 0.60 or less has a bad CU.

In an alternative embodiment, the CU value may be determined by the ECU using the following calculation rule:

$${CU}^{′′′′} = 1 - \frac{max ( \{ {Z}_{n , n + 1} \} ) - min ( \{ {Z}_{n , n + 1} \} )}{max ( \{ {Z}_{n , n + 1} \} )} .$$

This embodiment has the same tendency as the above calculation rule for CU″ and CU′″. Here the scaling is not relative to average contact but relative to the best contact. It was observed that a CU″″ value of 0.72 refers to a mediocre CU, whereas a CU″ value of 0.67 or less has a bad CU. In some embodiments it might be advantageous to combine two or more of the described methods for calculating the contact uniformity CU.

In another embodiment, the ECU is configured to determine the CU value for the subgroup of electrodes based on the standard deviation of quasi-unipolar impedance measurement values of all electrodes of said subgroup or based on the minimum and the maximum of the quasi-unipolar impedance measurement values of all electrodes said subgroup. The quasi-unipolar impedance measurement values are determined by measuring one electrode against all electrodes of opposing polarity (e.g. electrode 1 versus all even electrodes). In order to determine the quasi-unipolar impedance value for all electrodes, the impedance of each electrode of the even group of the subgroup is measured against one pre-defined odd electrode with the number and the impedance of each electrode of the odd group of the subgroup is measured against one pre-defined even electrode. Accordingly, there is one impedance measurement value for each electrode n of the subgroup, as n is either odd or even. The CU values are determined as described in the above calculation rules for CU, CU′, CU″, CU′″, CU″″, wherein Z_{n,n+1} is replaced by the impedances of the respective electrode Zₙ. The above explanations with regard to bipolar impedance measurements for determination of CU value apply for the quasi-unipolar impedance value, as well. However, if one even and one odd electrode are in close proximity (or in worst case) in contact, all CU value determination is strongly influenced by this condition. Then, the measurement exhibits a bipolar character.

In an alternative to the embodiment as described above, the quasi-unipolar impedance measurement values are determined by measuring one electrode against a selected group of electrodes of opposing polarity (e.g. electrode 1 versus a selected group of even electrodes). In this embodiment, the electrodes of opposing polarity are grouped in at least two groups based on the distance to the one electrode. For each electrode at least two groups of opposing polarities may be defined. A first group of the at least two groups may comprise the electrodes of opposing polarity near-by to the one electrode. A second group of the at least two groups may comprise the electrodes of opposing polarity far away to the one electrode. In this embodiment the ECU is configured to determine the CU value for the subgroup of electrodes based on the standard deviation of quasi-unipolar impedance measurement values of all electrodes of said subgroup or based on the minimum and the maximum of the quasi-unipolar impedance measurement values of all electrodes said subgroup, whereby the quasi-unipolar impedance values are determined by measuring one electrode against all electrodes of the second group. Quasi-unipolar impedance measurements based on the first group comprising the near-by electrodes are not considered in this embodiment. Thereby a bipolar character of the quasi-unipolar measurements is avoided.

With regard to above measurements in one embodiment, the measurement unit is configured such that the frequency for determination of quasi-unipolar or bipolar impedance measurement values of the subgroup of electrodes is between 1 kHz and 1 MHz and/or such that the voltage amplitude of the pulses is between 1V and 1 kV, in particular between 10V and 700V, in particular between 100V and 500V.

In another embodiment, it is considered that impedance measurements are also sensitive to electrode design (e.g. length, radius, spacing). These parameters can vary on the catheter (e.g. distal electrodes shorter than proximal electrodes). Since the designs are known, the algorithm realized by the ECU accounts for the differences by an additional scaling factor. For instance, measurements between 4 mm and 6 mm electrodes are treated differently than for 6 mm to 8 mm electrodes (comparably lower impedance expected). So, impedance measurements may be scaled using that information. The information on catheter design may also be used to simplify the algorithm. If electrodes cannot touch or come close by design (e.g. they are on opposite sides of one spline) then this reduces the number of measurements needed for the algorithm. If its desired to have the generator working with unknown catheters, a pretesting method may be implemented. This could be realized by asking the HCP to place the catheter in the blood (no wall contact). By pretesting the catheter measures impedance of pairs of adjoining electrodes and the difference in values can be ascribed to the catheter design. Furthermore, this possibly allows for a reduction of impedance measurements as this precheck may already identify electrodes that are far apart and will not touch even if the catheter is compressed and/or twisted. Alternatively, the measurements may be performed constantly and the lowest values (contact increases impedance) will be taken for the scaling process.

For providing the measurements the system is configured to select electrodes (e.g. using a multiplexer). If needed, accuracy can be increased by averaging multiple measurements and/or perform measurements synced to the QRS complex of the patient's heart in order to reduce artefacts from the beating heart. However, the advantage of improved data quality needs to be balanced with the inherent prolongation of the measurement duration.

In one embodiment, beyond measurements the catheter also allows to select electrode groups to apply pulsed energy. This feature may be achieved, as an example, by interaction with a user interface located on the catheter front panel. The HCP may proactively select groups of therapy-providing electrodes. The selection may be made such that electrodes with a high AR index or with an uneven CU value are placed in different therapy groups.

According to another aspect of the present invention, at least the above problem is solved by a method for assessment of positions and/or configuration of a plurality of electrodes of an ablation catheter for treatment of patient tissue by delivery of high-voltage pulses comprising a catheter shaft and an ablation portion, wherein the ablation portion is arranged at a distal end of the catheter shaft with the plurality of electrodes accommodated along the ablation portion, wherein each of the plurality of electrodes is electrically connected to a measurement unit through the catheter shaft, wherein the measurement unit performs measurements using an energy source thereby determining measurement values, in particular bipolar impedance measurement values of electrode pairs of a subgroup of the plurality of electrodes and/or quasi-unipolar impedance measurement values of a subgroup of the plurality of electrodes and/or current measurement values of a subgroup of the plurality of electrodes, wherein the subgroup is formed by all electrodes or a part of the plurality of electrodes, respectively, whereby the impedance and/or current measurement values may be determined as response to an alternating voltage and/or at least one voltage pulse, wherein an electronic control unit (ECU, 70) may be arranged proximal to or at the proximal end of the catheter, wherein the measurement unit may be connected to or integrated within the ECU, wherein said measurement values are transmitted to the ECU which receives and analyzes said measurement values as well as determines arcing risk (AR) and/or a contact uniformity (CU) and/or an impedance uniformity (IU) indexes based on said measurement values.

The arcing risk and/or the contact uniformity indexes may be based on the impedance measurement values and/or impedance for said electrodes. The impedance uniformity indexes may be based on the current measurements.

The above method (or algorithm) was already described with regard to the system above. It is therefore referred to the above explanation. The above method may be provided prior a PFA treatment using the electrodes of the ablation portion of the same system or in between two cycles of such treatment.

In one embodiment of the method, the measurement unit determines at least one current measurement value for each of the subgroup of the plurality of electrodes by measuring the respective current value of one or several of rectangular, sinusoidal, tooth or similar shaped voltage pulses, wherein one impedance value is determined from said determined current measurement values for each electrode of the subgroup of electrodes.

In one embodiment of the method, the ECU determines an impedance uniformity (IU) of two groups d,p of the subgroup of electrodes, wherein

$$IU = 1 - \frac{1}{2} ( \frac{σ ( \{ {Z}_{d} \} )}{μ ( \{ {Z}_{d} \} )} + \frac{σ ( \{ {Z}_{p} \} )}{μ ( \{ {Z}_{p} \} )} ) \vert ,$$

wherein σ({Z_{d,p}}) is the standard deviation and μ({Z_{d,p}}) the mean value of the determined impedances of the electrodes of the respective group.

In one embodiment of the method, the ECU determines the AR index for a particular electrode pair x,y from the bipolar impedance measurement values of the particular electrode pair x,y from the subgroup of electrodes scaled by the minimum of bipolar impedance measurement values of the respective electrodes with their adjoining electrodes of the subgroup.

In one embodiment of the method, the ECU determines the CU value for the subgroup of electrodes based on the standard deviation of the bipolar impedance measurement values of the pairs of adjoining electrodes of said subgroup or based on the minimum and the maximum of the bipolar impedance measurement values of the pairs adjoining electrodes of said subgroup and/or determines the CU value for the subgroup of electrodes based on the standard deviation of the quasi-unipolar impedance measurement values of all electrodes of said subgroup or based on the minimum and the maximum of the quasi-unipolar impedance measurement values of all electrodes of said subgroup.

In one embodiment of the method, the ECU determines an overall risk for arcing for all electrodes of the subgroup based on a maximum of the AR index of all electrode pairs of the subgroup.

In one embodiment, the ablation portion comprises at least one loop section, for example at least two loop sections forming a three-dimensional spiral. The at least two loop sections may be arranged as a continuous or discontinuous spiral. In this case, the beginning and the end of each loop section could be arranged either in the same or in a different plane with respect to the central axis of the three-dimensional spiral. In addition, the at least two loop sections itself could be arranged either in the same or in a different plane with respect to the central axis of the three-dimensional spiral. An example of at least two loop sections forming a continuous spiral is shown in FIG. 1, whereby the beginning and the end of each loop section is arranged in a different plane with respect to the central axis of the three-dimensional axis and whereby the at least two loops are arranged in different planes with respect to the three-dimensional axis.

In one embodiment, the diameters of two neighboring loop sections increase into the direction of the distal end of ablation portion forming a plunger type ablation catheter. The plunger type ablation catheter may be used for ablation in the ventricles or in the atrial area of the posterior left atrium. Alternatively, the diameters of two neighboring loop section decrease into the direction of the distal end of the ablation portion forming a corkscrew type ablation catheter. The corkscrew type ablation catheter may be used for ablation in the area of the atrial end of the PV. The diameters of loop sections may be, for example, between 10 mm and 40 mm. More specifically, if used in the left atrium, the widest loop section may have a diameter between 20-35 mm, preferably between 25-32 mm. The smallest diameter can be 12-22 mm, preferably 15-20 mm. The diameter is measured from both inner surfaces of opposite loop sections. For both areas, the form of the ablation portion is adapted to the specific form of the respective area to be ablated.

It is also within the scope of the present invention that the ablation portion may comprise a plurality of separate mapping electrodes, the mapping electrodes being configured for receiving electrical signals, e.g. electrical or biopotential, from ventricular, vascular or atrial tissue. Alternatively, the electrodes used for ablation in the ablation mode may be used for mapping, namely receiving electrical biosignals, e.g. acquiring electrical or biopotential, from ventricular, vascular or atrial tissue. During ablation these electrodes are in the ablation mode. This may enable mapping and ablation with a single ablation catheter for PVI as well as ablating some non-PV triggers for AF patients.

For example, in an embodiment, an additional loop section of the plurality of loop sections may exhibit a plurality of mapping electrodes (electrodes exclusively used for mapping). Additionally or alternatively, mapping electrodes may also be arranged—in addition to the ablation electrodes—on one or both of the two neighboring loop sections. A plurality of mapping electrodes may also be incorporated distal to the plurality of ablation electrodes, or medially within two ablation electrodes, e.g. between two ablation electrodes (along the respective loop section). Furthermore, the third loop section may comprise ablation electrodes in addition to or instead of the mapping electrodes.

In one embodiment, the ablation portion, and in particular the loop sections, may comprise a shape memory material. Preferably, the shape memory material is a super-elastic material (such as a super-elastic alloy), which is to say that the material is elastic and has a shape memory property. For example, Nitinol is a biocompatible super-elastic alloy that is suitable for the present purpose. In one variant, the ablation portion, and in particular the loop sections, may comprise an inner support element, such as an inner support wire, having a shape memory or super-elastic property. The shape memory support wire may have various stiffness and cross-sectional shapes in different sections. The inner support structure maintains the architecture and design integrity of the ablation portion and extends along at least a section of the ablation portion. The inner support structure may be realized as a Nitinol wire (for example a round, rectangular, square wire with variable cross section or tapered). In addition, this support structure comprises insulated with material, for example Parylene, Polyimide, Teflon at the outer surface of the wire. Further, the wire of the ablation portion may have sections with different diameter or cross-sectional shape in order to provide different stiffness.

In an embodiment, the ablation catheter may further comprise a steerable delivery sheath. Thus, in operation, a position of the ablation portion may be easily adjusted at the target tissue until the contact of each ablation electrode is satisfied.

In one embodiment, the catheter shaft comprises at least two lumens separated by a material with a dielectric strength greater than a dielectric threshold suitable to withstand high-voltage PF pulses used with the above and below described system/catheter, for example with high-voltage PF pulses having an amplitude greater than 1 kV, greater than 2.5 kV or between 2.5 kV and 3.5 kV. Such material may be, for example, a polymer film, in particular a Polyimide film (e.g. Kapton® film) provided in form of tubing or a layer received by dipping. It has a dielectric strength of 160 kV/mm. The thickness of the polymer film (Polyimide layer) may be chosen in the range of 0.012 mm to 0.125 mm, for example. In this embodiment, the first lumen of the at least two lumens is configured to retain at least two electrical conductors which are connected with electrodes providing the same first polarity and wherein the second lumen of the at last two lumens different from the first lumen is configured to retain at least two electrical conductors which are connected with electrodes providing the same second polarity different from the first polarity. This embodiment allows to reduce the diameter of the catheter shaft as the isolation of each electrical conductor is not necessary and to provide necessary safety with regard to arcing at the same time.

In one embodiment, the catheter shaft may have an overall length greater than 1 m from the handle to the distal tip of the ablation portion.

In one embodiment, at least two of the plurality of electrodes of the ablation portion are adapted to deliver high voltage unipolar PF energy or bipolar PF energy or a combination of unipolar and bipolar PF energy as described below. A schematic example of an applicable waveform is shown in FIG. 13. Such waveforms, in combination with the loop structures described above, ensure one-shot application of electrical fields that are high and long enough to generate therapeutic effects capable of creating moats of conduction block, yet lower and shorter than ionization thresholds so to avoid arcing. The PFA pulses can be delivered gated by the QRS complex of the cardiac cycle. Alternatively, when ablation targets regions remote from ventricles, PFA pulses may be delivered asynchronously, without QRS gating. The electronic control unit is adapted to switch between unipolar PF energy and bipolar PF energy supply mode.

In another embodiment, the distal tip of the ablation portion is connected with steering wires or center wire which may be manipulated from a handle element provided at the proximal end of the catheter shaft. Accordingly, the center wire may be connected to an actuation mechanism within the handle element. Along the ablation portion, the center wire approximately run along a longitudinal axis of the catheter shaft. A steering plate, steering ring, or other known steering structures may be placed at the distal end of the catheter shaft, which connects to the distal spiral, or multiple loop, ablation section. The center wire connects to said steering structure. The center wire may be manipulated such that a longitudinal length of the ablation portion (i.e. its length along the longitudinal axis of the three-dimensional spiral/multiple loop structure) or the loop sections may be steered towards tissue targets, according to the therapeutic needs.

In one embodiment, the electrodes are distributed along the at least two loops in a way, that the angular separation between the most distal and the most proximal electrode is at least 360°. The angular separation is determined by the angle between the most distal electrode, the catheter axis and the most proximal electrode. Furthermore, electrodes may be distributed along the at least two loops in a way, that the longitudinal separation between the most distal and the most proximal electrode is at least 5 mm. The longitudinal separation is understood as distance along the catheter axis between the most distal electrode and the most proximal electrode.

The above method is, for example, realized as a computer program (to be executed within the system, in particular within its ECU) which is a combination of above and below specified (computer) instructions and data definitions that enable computer hardware or a communication system to perform computational or control functions and/or operations, or which is a syntactic unit that conforms to the rules of a particular programming language and that is composed of declarations and statements or instructions needed for an above and below specified function, task, or problem solution.

Furthermore, a computer program product is disclosed comprising instructions which, when executed by a processor, for example a processor of the ECU, cause the processor to perform the steps of the above defined method. Accordingly, a computer readable data carrier storing such computer program product is described. The computer program product may be a software routine, e.g. related to hardware support means within the processor of the ECU.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein schematically and exemplarily,

FIG. 1 depicts a distal end of a first embodiment of an ablation catheter in a perspective side view;

FIG. 2 illustrates a delivery path for an ablation catheter leading to a pulmonary vein ostium of a human heart;

FIGS. 3-3A show part of the electric control of the electrode leads for the embodiment of the ablation catheter of FIG. 1;

FIG. 4 depicts the distal end of the ablation catheter of FIG. 1 with electrode numbering in a top view;

FIGS. 5 and 6 show matrices containing AR indexes for each electrode pair and the impedance values of the ablation catheter of FIG. 1 for a saline position of the ablation portion;

FIG. 7 shows the ablation portion of the ablation catheter of FIG. 1 pressed to chicken heart tissue in a top view;

FIGS. 8 and 9 show matrices containing AR indexes for each electrode pair and the CU value of the ablation catheter of FIG. 1 in the position shown in FIG. 7;

FIG. 10 shows another position of the ablation portion of the ablation catheter of FIG. 1 pressed to chicken heart tissue in a top view;

FIGS. 11-12 show matrices containing AR indexes for each electrode pair and the CU value of the ablation catheter of FIG. 1 in the position shown in FIG. 10;

FIG. 13 depicts a schematic example of an applicable PFA waveform;

FIG. 14 shows another position of the ablation portion of the ablation catheter of FIG. 1 pressed to a chicken heart tissue in a top view;

FIG. 15 shows a matrix containing impedance values and an AR index (flagged) for the electrode pairs 1,2; 2,3; 2,9; 8,9; 9,10, respectively of the ablation catheter of FIG. 1 in the position shown in FIG. 14;

FIG. 16 shows another position of the ablation portion of the ablation catheter of FIG. 1 pressed to a chicken heart tissue in a top view;

FIG. 17 shows a matrix containing impedance values and an AR index (flagged) for the electrode pairs 1,2; 2,3; 2,9; 8,9; 9,10, respectively of the ablation catheter of FIG. 1 in the position shown in FIG. 16;

FIG. 18 shows another position of the ablation portion of the ablation catheter of FIG. 1 pressed to a chicken heart tissue in a top view;

FIG. 19 shows a matrix containing impedance values and an AR index (flagged) for the electrode pairs 1,2; 2,3; 2,9; 8,9; 9,10, respectively of the ablation catheter of FIG. 1 in the position shown in FIG. 18;

FIG. 20 shows another position of the ablation portion of the ablation catheter of FIG. 1 pressed to a chicken heart tissue in a top view;

FIG. 20A shows another position of the ablation portion of the ablation catheter of FIG. 1 pressed to a chicken heart tissue in a top view;

FIG. 21 shows a matrix containing impedance values for the adjoining electrode pairs derived from a bipolar measurement and a CU value of the ablation catheter of FIG. 1 in the position shown in FIG. 20 calculated from these impedance values;

FIG. 21A shows a matrix containing impedance values for the adjoining electrode pairs derived from a bipolar measurement and a CU value of the ablation catheter of FIG. 1 in the position shown in FIG. 20A calculated from these impedance values;

FIG. 22 shows a table containing impedance values derived from a quasi-unipolar measurement at 500 kHz and a CU value calculated from these impedance values of the ablation catheter of FIG. 1 in the position shown in FIG. 20;

FIG. 22A shows a table containing impedance values derived from a quasi-unipolar measurement at 500 kHz and a CU value calculated from these impedance values of the ablation catheter of FIG. 1 in the position shown in FIG. 20A;

FIGS. 23-25 visualize three different pulse shapes for current measurements at each individual electrode;

FIGS. 26-29 show four different positions of the ablation catheter of FIG. 1, partly with respect to a chicken heart tissue in saline;

FIG. 30 shows a bar diagram containing impedance values determined for the four positions of FIGS. 26 to 29 with respect to each electrode of the ablation portion of the ablation catheter of FIG. 1;

FIG. 31 shows a position of the ablation catheter of FIG. 1 within a heart of an 80 kg pig;

FIG. 32 shows a bar diagram containing impedance values determined for the position of the catheter depicted in FIG. 31 with respect to each electrode of the ablation portion of the catheter;

FIG. 33 visualizes a flowchart for the use of a PFA catheter including PFA precheck determining AR indexes and CU value in order to treat paroxysmal atrial fibrillation; and

FIGS. 34-35 show examples of visualization of impedance values for electrodes of an ablation section of an ablation catheter similar to the one of FIG. 1.

### DETAILED DESCRIPTION

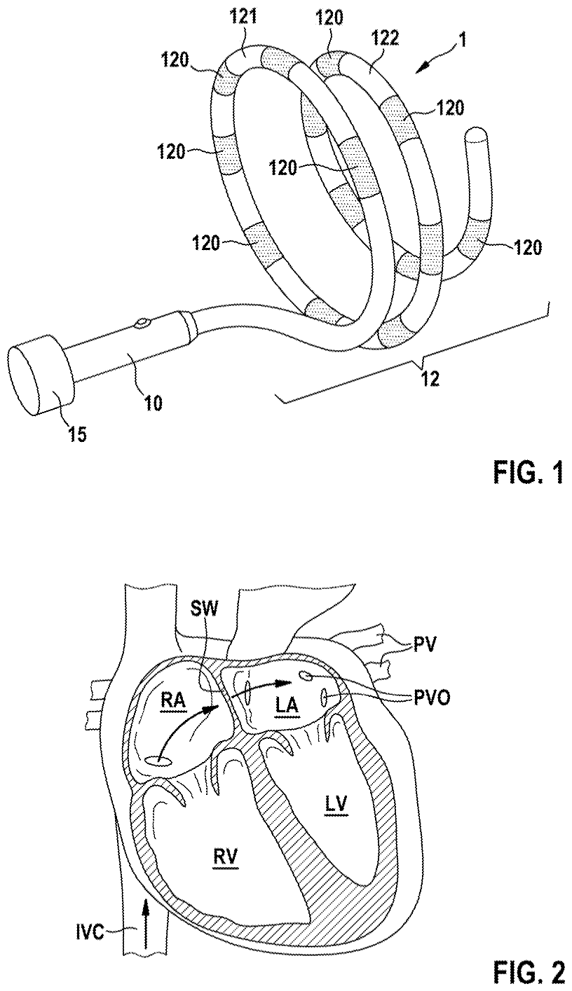

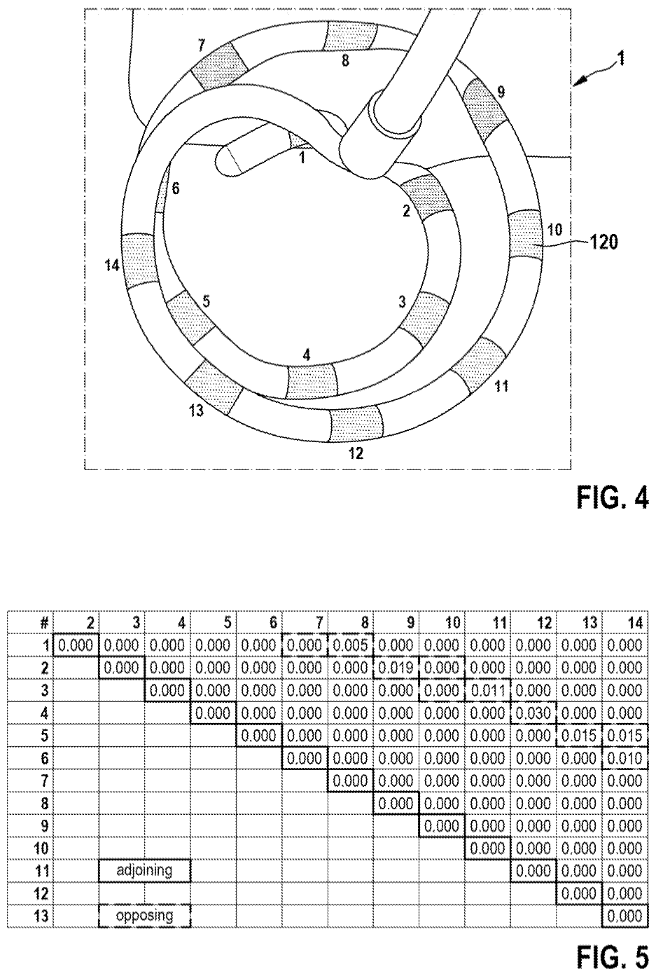

FIGS. 1 and 4 illustrate a distal portion of an ablation catheter 1 in accordance with a first embodiment. The ablation catheter may be used for PFA, when used with the PFA generator and accessories, and is indicated for use in cardiac electrophysiological mapping (stimulation and recording) and in high-voltage, pulsed-field cardiac ablation. Peak voltages are, for example, without limitation, +/−1 kV to 3 kV with a pulse width of up to 30 μs. Higher peak voltages (e.g. up to 10 kV) may be used provided the pulse duration is correspondingly shorter (e.g. 0.5 μs). The catheter 1 has an elongated circular catheter shaft 10, which may connect with a handle comprising a steering mechanism at a proximal end (not illustrated). As a result, the catheter may control deflections of the depicted distal section carrying the ablation electrodes.

At the illustrated distal end of the catheter shaft 10 an ablation portion 12 is arranged, which comprises a plurality of loop sections 121, 122. The concept of loop sections includes embodiments that use continuous loops or spirals configurations. The catheter shaft may have an effective length of approximately 115 cm from the distal tip of the ablation portion 12. Each of a first loop section 121 and a neighboring second loop section 122 exhibits ablation electrodes 120 (altogether, for example, 14 electrodes), which are configured for delivering energy to tissue. Although two loops are illustrated in FIG. 1, more can be used. It is preferred that at least a partial third loop is used in order to provide sufficient overlap among resulting ablation zones. Said overlap would increase chances of achieving a conduction block moat without drops in lesion continuity, contiguity or transmurality. The distal section comprises at least 45° of overlap of a 3^{rd} loop section with the previous two sections. In particular, the ablation catheter 1 may be configured for delivering an electrical high voltage PFA signal to tissue via the ablation electrodes 120. For example, the ablation electrodes 120 may consist of or comprise gold and/or a platinum/iridium alloy. Alternatively, electrodes 120 from different loop sections may be positioned so that electrodes of same polarity are aligned. However, dependent on the form of the patient's tissue and the position of the ablation portion 12, electrodes of opposite polarities may collide when the spiral catheter is compressed thereby causing arcing and/or the contact of the electrodes with the patient's tissue may not be uniform. In the exemplary embodiment illustrated in FIG. 1, the ablation electrodes 120 of the second loop section 122 are arranged partly in a staggered manner with respect to the ablation electrodes 120 of the first loop section 121.

In order to address measurement values to the different electrodes 120, the electrodes are consecutively numbered as shown in FIG. 4 (see numbers at the electrodes). The most distal electrode has the number 1, whereas the most proximal electrode is denoted with number 14. Different numbering is possible, as well.

The loop sections 121, 122 may further exhibit a plurality of mapping electrodes, which are configured for receiving electrical signals from tissue.

Together, the loop sections 121, 122 form a three-dimensional spiral, which form a corkscrew-similar form. Alternatively, they may form a plunger-like configuration or any other suitable 3-dimensional configuration (not shown).

The loop sections 121, 122 may comprise a shape memory material, for example, in the form of an inner structural support wire (not illustrated), for example a Nitinol wire as described above. In particular, the loop sections 121, 122 may have super-elastic properties.

The ablation portion 12 may be constrained into an essentially elongate shape for the purpose of delivery to a target region in the human body by means of a (fixed or steerable) delivery sheath 15, which may also be referred to as an introducer sheath. At the target position, upon exiting a distal end of the delivery sheath 15, the ablation portion 12 may then recoil to its original (biased) shape.

The length of each electrode 120 along the respective loop section 121, 122 is, for example, 4 mm. In general, the electrode length is in the range 1-10 mm, preferably 3-5 mm. The catheter shaft 10 size may be compatible with an 8.5 F ID sheath and may consist of radiopaque extrudable polymer and, if applicable, a polymer-reinforcing braid. In general, the size of the catheter shaft 10 may be compatible with a 7 F to 14 F ID sheath. The width between neighboring electrodes along the respective loop section may be chosen between 1 mm and 10 mm, preferably 3-6 mm, in order to provide a contiguous ablated area at the patient's tissue.

FIG. 2 schematically and exemplarily illustrates a delivery path for an ablation catheter 1 leading to a pulmonary vein ostium (PVO) of a human heart. For orientation, the inferior vena cava (IVC), the right atrium (RA), the right ventricle (RV), the left atrium (LA), the left ventricle (LV), as well as pulmonary veins (PV), each with a PVO, are shown. The large black arrows indicate a delivery path passing through the IVC, the RA, transeptally through the septal wall (SW), and into the LA. Finally, using appropriate deflection means, catheter 1 is steered to PVO regions. There, the corkscrew type ablation catheter may be used for ablation in the area of the atrial end of the pulmonary vein close to PVO. The form of the ablation portion 12 is configured such that it fits to the dimensions of the targeted PVO. Alternatively, corkscrew-type catheters may be used to ablate at the SVC or at Appendages, such as the left or right atrial appendages (LAA or RAA).

Reliable full ablation along a whole circumference is achieved with the first embodiment of the ablation catheter shown in FIGS. 1 and 4 at their respective position within the heart or the vein to which the form is adapted. A small compression of the ablation portion 12 of the respective catheter 1 may be possible during ablation into the direction of the longitudinal axis of the spiral.

The ablation procedure using one of the ablation catheters 1 may start after the ablation portion 12 is in the correct position relative to the targeted tissue, for example at a PVO. The assessment of the position and/or configuration of the ablation electrodes 120 is provided prior and/or between two ablation steps (if applicable) and is explained in more detail below. The ablation electrodes 120 will provide pulsed electric RF field in a unipolar or bipolar arrangement. Peak voltages are, for example, without limitation, +/−1 kV to 3 kV with a pulse width of up to 30 μs. Higher peak voltages (e.g. up to 10 kV) may be used provided the pulse duration is correspondingly shorter (e.g. 0.5 μs). The pulse width may be 12 μs (between 0.5-30 μs) forming a pulse train comprising up to 500 pulses/train.

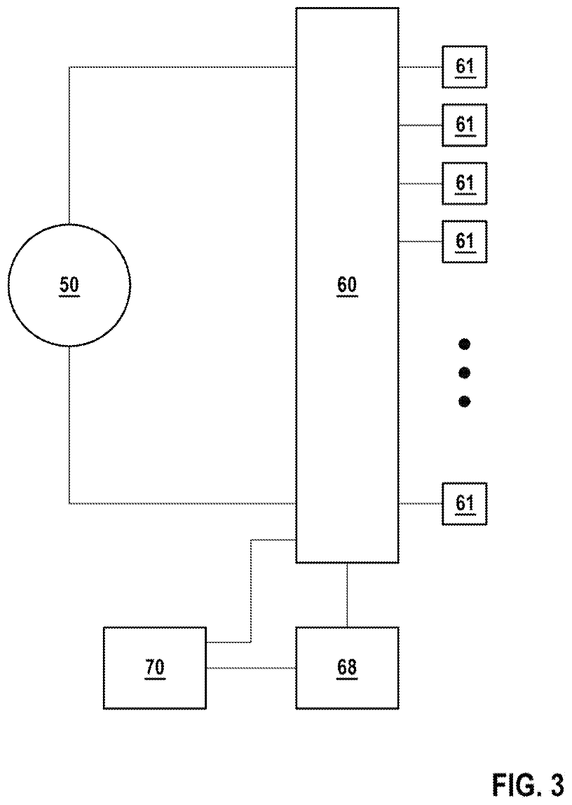

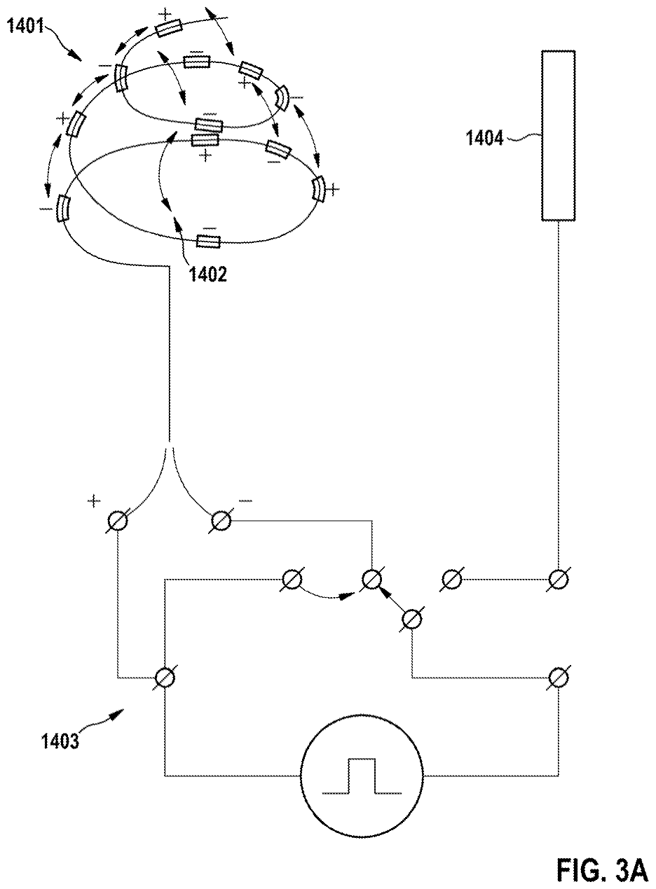

The electric field generation (in particular voltage, current and impedance) is monitored by an electronic control unit (ECU) 70 which is connected to the leads 61 of the electrodes 120 and produced by a waveform generator 50 (see FIG. 3). FIG. 3A also shows connectivity that can be used to generate unipolar or bipolar electric fields. ECUs in FIGS. 3 and 3A may control application of PFA fields. FIG. 3A illustrates a catheter 1401 (such was the one with reference number 1 from FIGS. 1 and 4) with its electrodes driven by ECU 1403. ECU 1403 can be controlled to deliver field vectors 1402 that cover the tissue zone in between catheter 1401 spiral arms/loops. By doing so, the AR index may be determined. In order to provide quasi-unipolar measurements, the PFA generator may be connected to one of the electrodes as the reference electrode instead of to the grounding pad 1404.

In order to assess the positions and/or configuration of the electrodes 120 with regard to each other and the targeted tissue, the ablation catheter further comprises a measurement unit 68 which is connected to the ECU 70 and a switch unit 60 with the waveform generator 50. The measurement unit 68 is configured to measure peak current and peak voltage as well as impedance at the respective electrode lead 61 and transmit these data to the ECU for further analysis. Further, the measurement unit 68 provides the electrodes 120 at the respective lead(s) 61 with pre-defined measurement signals (current or voltage pulses) via the waveform generator 50 in order to measure the above-mentioned parameter.

In the bipolar arrangement neighboring (adjoining) electrodes 120 may be paired along the loop sections 121, 122, across two neighboring loop sections 121 and 122 or any other pre-defined pair combination, in particular for impedance determination for AR value and/or CU value. Further, the electrodes 120 may be used in a unipolar arrangement. In this case, a ground pad 1404 may be provided at the surface of the patient's body. Alternatively, one of the non-adjacent electrodes 120 may be used as reference electrode thereby forming a quasi-unipolar arrangement.

In order to switch between different bipolar arrangements or between unipolar and bipolar arrangement, the ablation catheter 1 may comprise a switch unit 60 connected to and controlled by the ECU 70. The switch unit 60 provides the respective phase of the pulsed electric field provided by the waveform generator 50 to the predefined electrode lead 61 and thereby to the predefined electrode 120 wherein each electrode lead 61 is electrically connected to one particular electrode 120 at the ablation portion 12. The switch unit 60 comprises a switch matrix and may realize any configuration of phase distribution, for example, such that two neighboring electrodes along the loop sections, across the loop sections and any other electrodes are paired. The switching signal and configuration information is provided by the ECU 70. ECU 70 further may provide data processing of electrical or biopotential data or impedance data acquired the electrodes of ablation catheter 1. As indicated above mapping electrodes located in the ablation portions 12 may comprise mapping electrodes for determining the electrical potential of the surrounding tissue in order to observe the ablation progress at pre-defined time points during ablation procedure. Alternatively, the ablation electrodes 120 may be switched into the mapping mode and back into the ablation mode.

As indicated in the general description, prior ablation treatment and/or between ablation treatment steps the AR value and CU value are determined in order to assess the positions of the electrodes 120 and/or their configuration with regard to each other and/or with regard to the tissue under treatment.

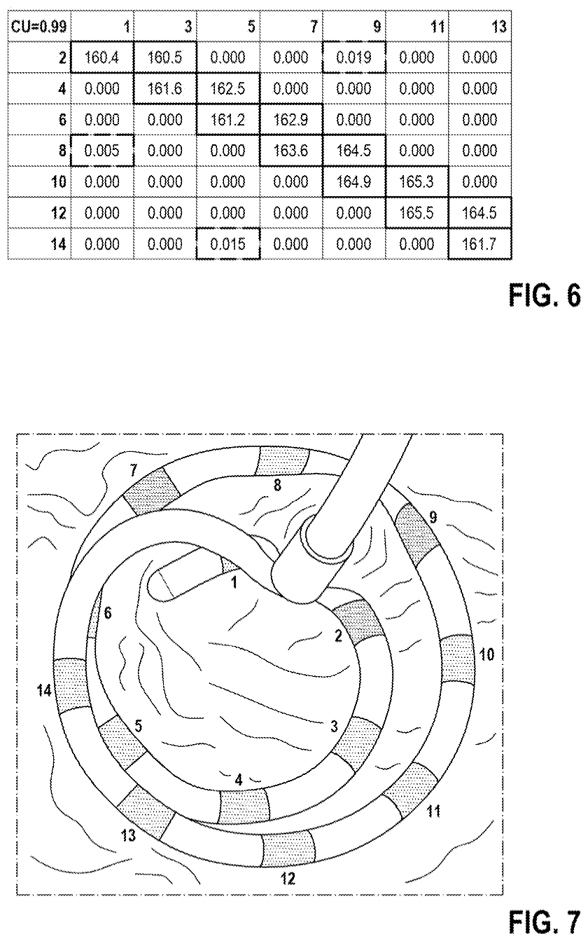

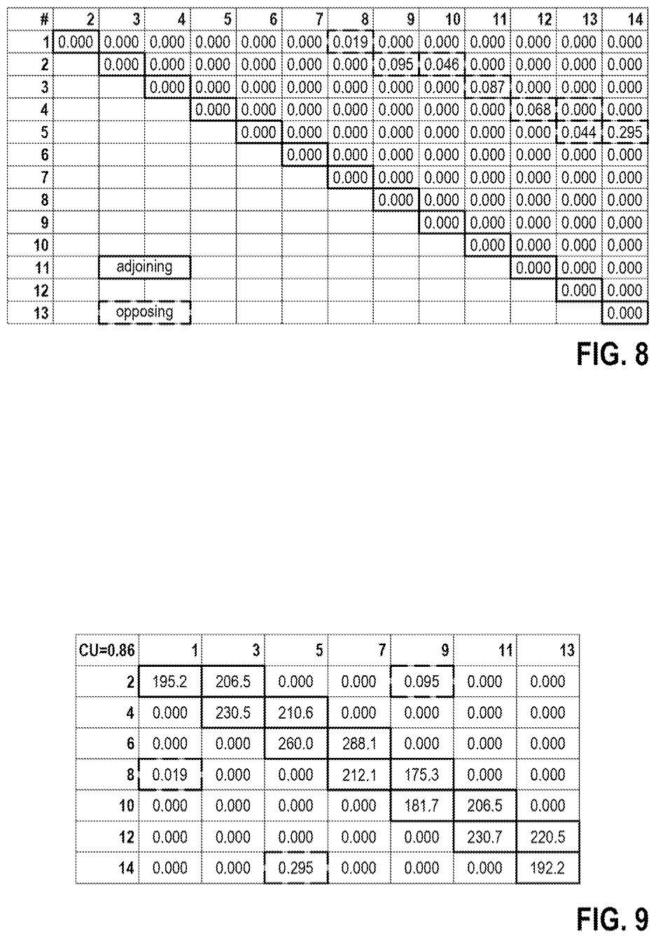

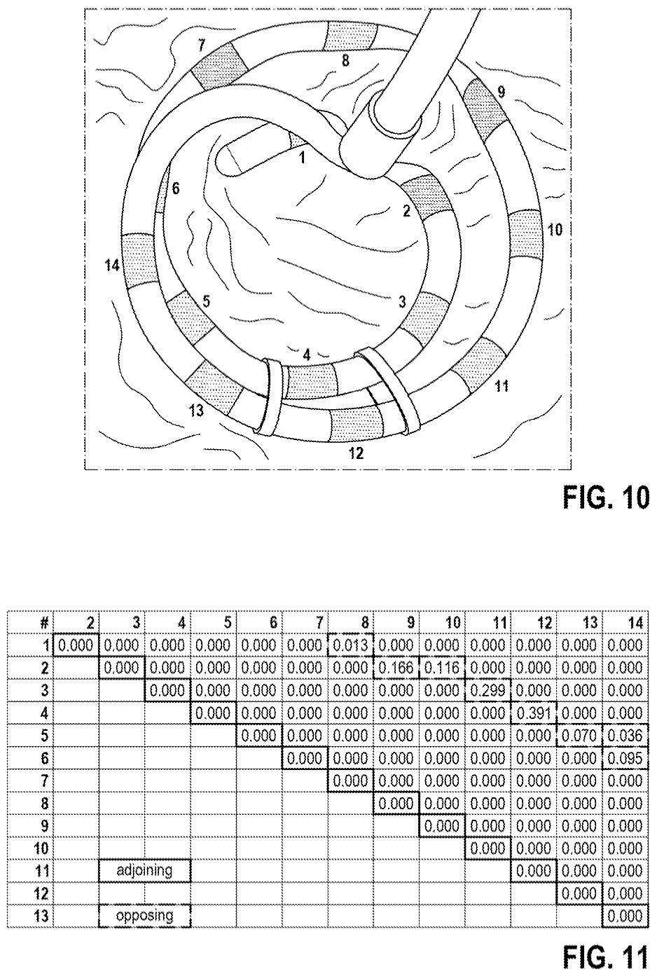

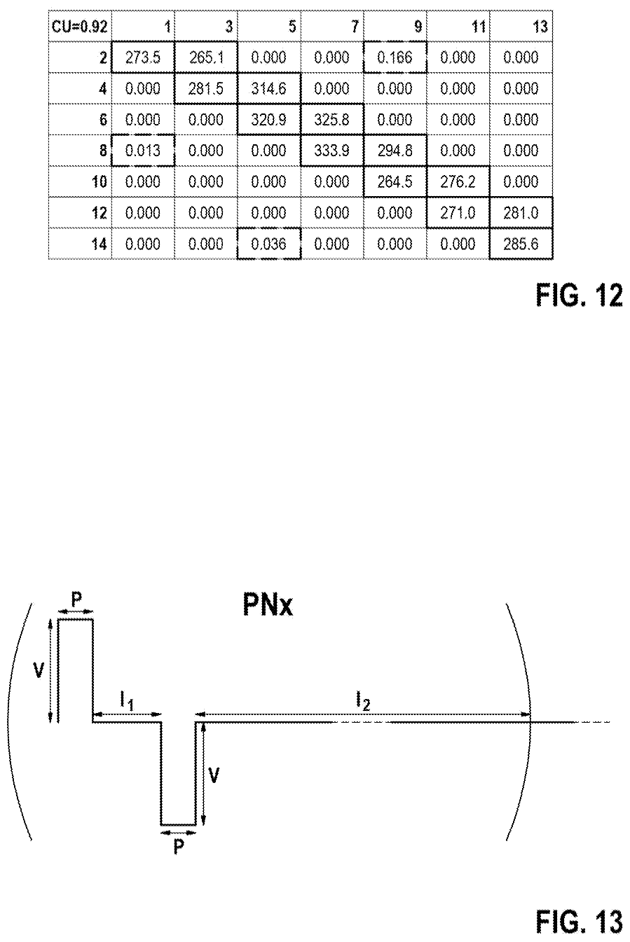

In the first example, the ablation catheter of FIGS. 1 and 4 is measured with regard to the impedance of all pairs of the 14 electrodes in saline (for comparison), a first position axially pressed to a chicken heart tissue (see FIG. 7) and in a second position axially pressed to a chicken heart tissue wherein black rubber bands keep the electrodes 4 and 12 close to each other (see FIG. 10). The matrices of FIGS. 5 and 6 belong to the saline configuration, the matrices of FIGS. 8 and 9 to the position shown in FIG. 7 and the matrices of FIGS. 11 and 12 to the position shown in FIG. 10.

For example, AC voltage signals with a frequency of 500 kHz with a peak voltage (amplitude) of 1 V are chosen. The matrices of FIGS. 5, 8 and 11 show the AR index calculated from the bipolar impedance measurement values Z_{x,y} of the electrode pair x,y. The number of the electrodes of the particular electrode pair can be found in the respective header line and the first row. The value at the row-line-intersection contains the AR index of the respective electrode pair x,y determined from the impedance measurement values for 500 kHz. The AR index is calculated using the formula

$${AR}_{x , y} = 1 - \frac{{Z}_{x , y}}{min ( {Z}_{x - 1 , x} , {Z}_{x , x + 1} , {Z}_{y - 1 , y} , {Z}_{y , y + 1} )} .$$

All AR index values are zero or close to zero for the saline configuration. No risk or arcing exists since all electrodes have a sufficient distance to each other.

In contrast, with regard to the ablation portion position of FIG. 7 it is apparent that the AR index of the electrode pair 5, 14 is considerable higher than the other AR indexes. In FIG. 7 it appears, that these electrodes are the only ones which are close to each other—there is an arcing risk with regard to these electrodes and repositioning is needed.

The matrix of FIG. 11 contains the AR index values calculated in a similar way for the configuration of FIG. 10 and a frequency of 500 kHz. It is apparent that in particular the electrode pairs 3, 11 and 4, 12 show considerable higher AR index values than any other AR index value of this matrix. For these pairs a risk for arcing exists, if the electrodes of these pairs would be at different polarities.

In another representation shown in FIGS. 6, 9 and 12 the calculated AR indexes of the respective electrode pairs (electrode numbers are shown in the header line and in the first row, formula see above) are provided for all electrode pairs but the adjoining electrode pairs (marked in the diagonal) for the respective ablation portion position. In the diagonal line the impedances of the adjoining electrode pairs are provided. In the matrix of FIG. 9 the AR index of the electrode pair 5 and 14 is highlighted since it indicates a high arcing risk (AR index >0.25). With regard to the third position (FIG. 10), in particular, the electrode pair 2, 9 has a higher arcing risk. Just for clarification, in this position the AR index values for the electrode pairs 4, 12 and 5, 13 are neglected since these electrodes share the same polarity and therefore no risk for arcing exists.

Further, the diagrams of FIGS. 6, 9 and 12 contain the CU value for the respective position in the upper left corner calculated from the following formula (see explanation above) and the measured bipolar impedances of the adjoining electrodes

$$CU = 1 - \frac{σ ( \{ {Z}_{n , n + 1} \} )}{μ ( \{ {Z}_{n , n + 1} \} )} .$$

It appears from the matrices in FIGS. 6, 9 and 12 that the contact uniformity of the position shown in FIG. 7 is better than of the position shown in FIG. 10 as the CU value is greater (0.92>0.86). The contact uniformity is best in the saline position (0.99)—if all electrodes without contact, i.e. all electrodes are floating in saline.

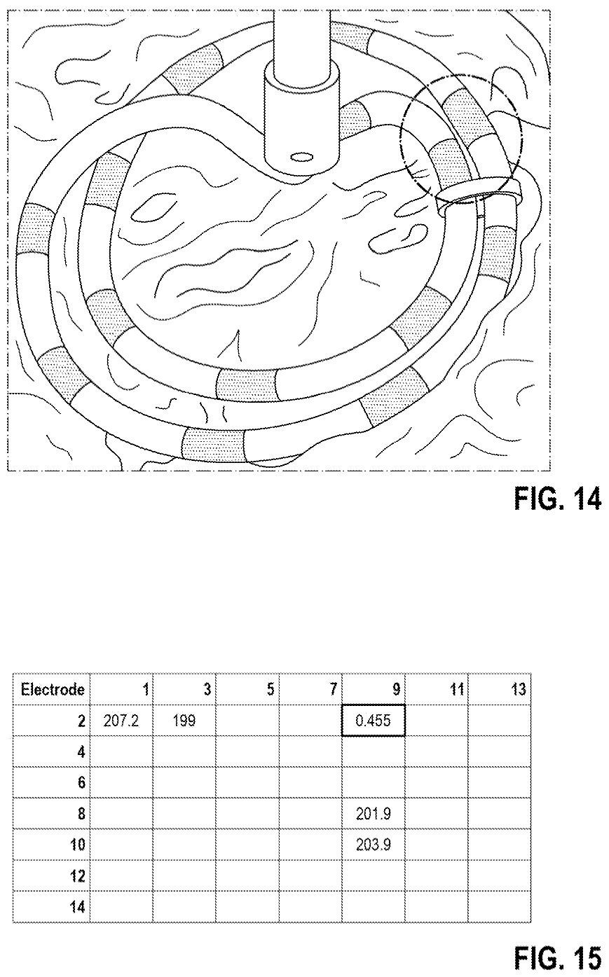

Further examples of ablation catheter positions pressed to a chicken heart are shown in the following FIGS. 14 to 19, wherein a profile shown in FIG. 13 is used as PFA protocol, wherein V=2.5 kV, P=3 μs, I₁=25 μs, and I₂=2 ms. Further, a pulse number PN=20 were chosen intentionally to provoke arcing.

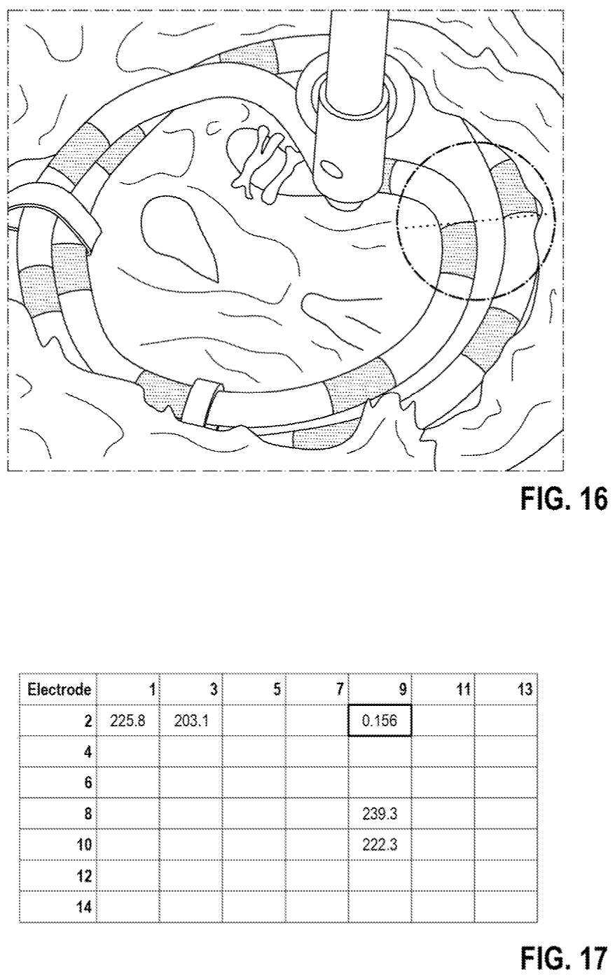

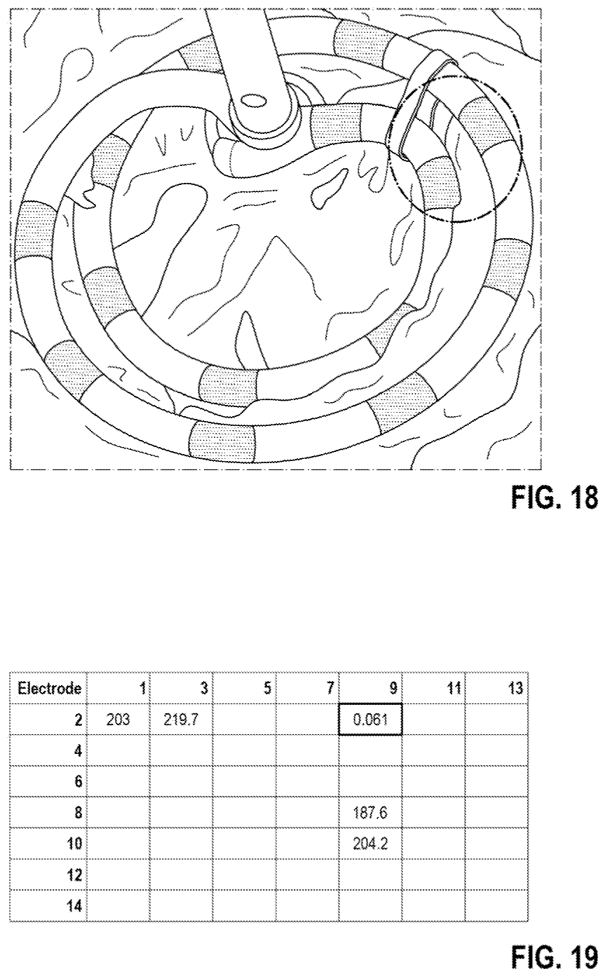

FIG. 14 shows a position of the ablation portion of the ablation catheter of FIGS. 1 and 4 in which the electrodes 2, 9 are in close proximity (see encircled area). Accordingly, the AR index of these electrodes is 0.455 indicating the high arcing risk (see matrix shown in FIG. 15). The arcing threshold was determined as 0.9 kV confirming the calculated AR index. FIG. 16 shows a position of the ablation portion of the ablation catheter of FIGS. 1 and 4 where electrodes 2, 9 do not overlap (see marked area, so-called edge-edge position). Accordingly, the AR index shown in FIG. 17 is lower than the one of FIG. 15. The lowest AR index may be found for the position of these electrodes 2,9 shown in FIG. 18 in which these electrodes are sufficiently far away thereby having a low arcing risk (see marked area). Accordingly, the AR index of this electrode pair 2, 9 is close to zero (see FIG. 19).

In another example, the CU value for two positions of the ablation catheter of FIGS. 1 and 4 is determined, in particular the CU value determined from bipolar impedance measurements of adjoining electrodes using formula (n=1 . . . 13)

$$CU = 1 - \frac{σ ( \{ {Z}_{n , n + 1} \} )}{μ ( \{ {Z}_{n , n + 1} \} )}$$

is compared with the CU value determined from quasi-unipolar impedance measurement values. For determination of the CU value for the quasi-unipolar impedance measurement values Zₙ in the above formula the parameter Z_{n,n+1} is replaced by Zₙ for the standard deviation and the mean value. In this case n=1 . . . 14. The quasi-unipolar impedance one electrode (e.g. electrode 1) is measured against all electrodes of opposing polarity (e.g. against all even electrodes, and electrode 2 against all odd electrodes).

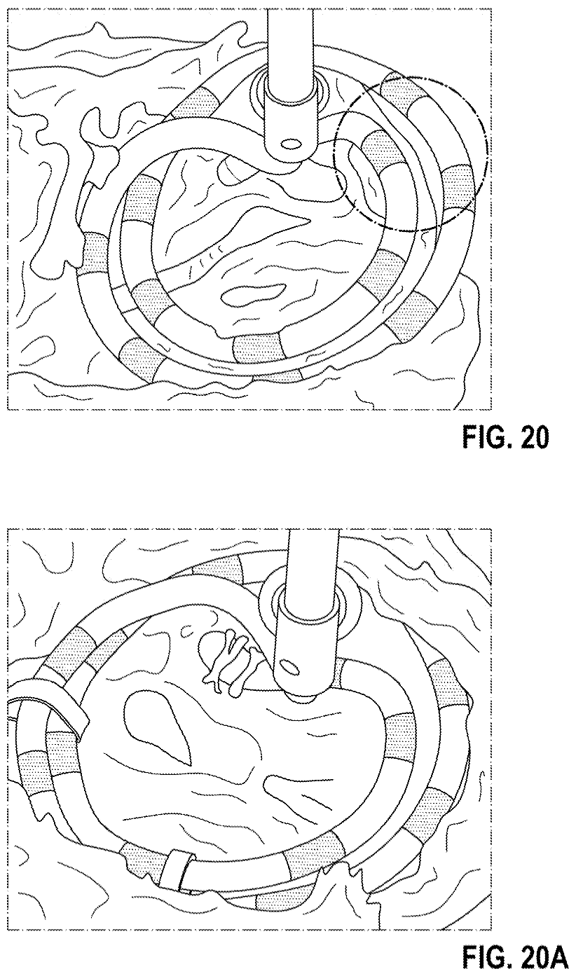

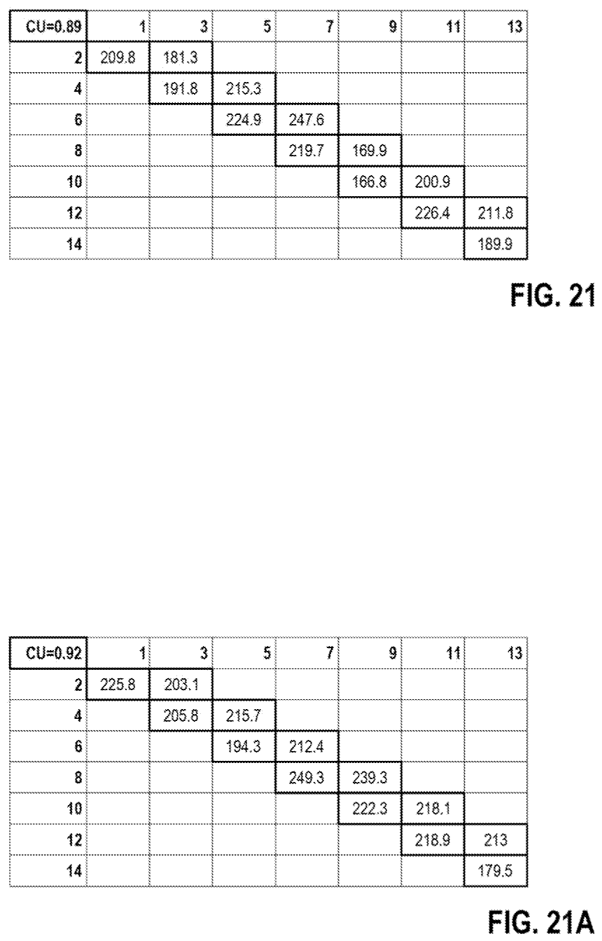

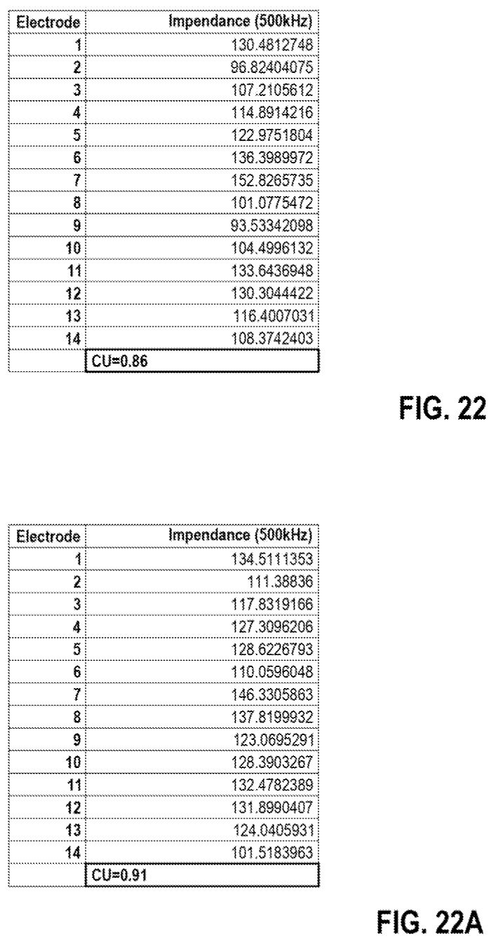

FIG. 20 shows a position in which three electrodes (2, 8, 9) are floating in saline while the others are in contact with the heart tissue. The CU value (bipolar, see FIG. 21) is 0.89 and the CU value (quasi-unipolar) is determined as 0.86 (see FIG. 22) which is comparably low thereby indicating bad contact uniformity. In contrast the position shown in FIG. 20A has all electrodes in contact with the chicken heart's tissue. Accordingly, CU value (bipolar, see FIG. 21A) is 0.92 and the CU value (quasi-unipolar) is determined as 0.91 (see FIG. 22A).

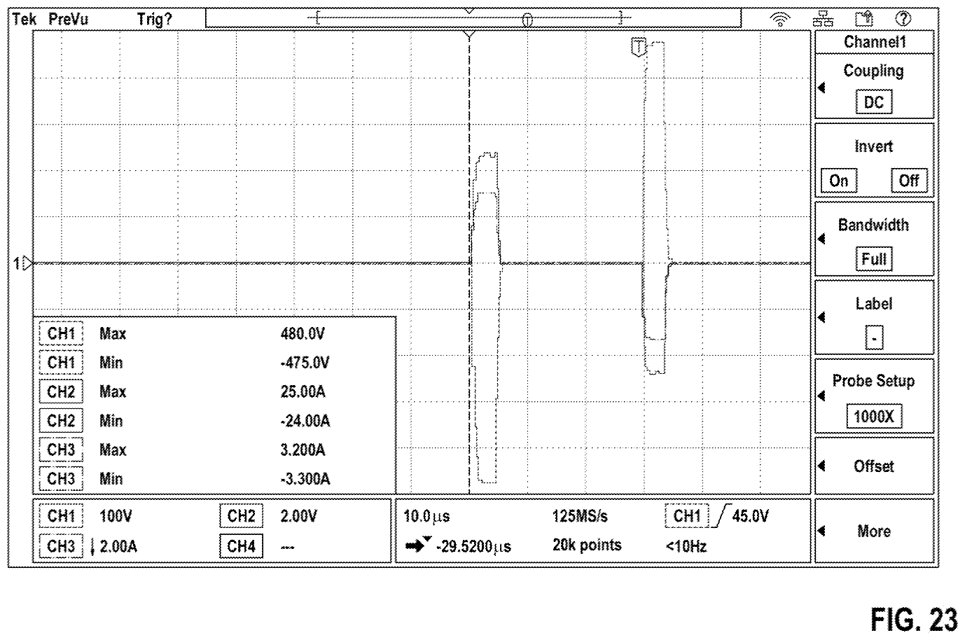

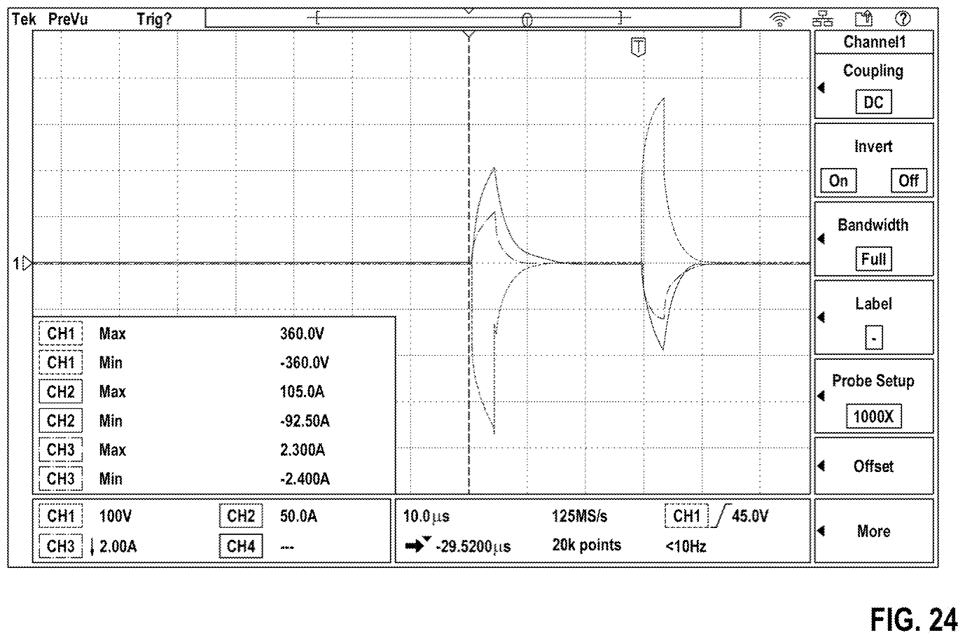

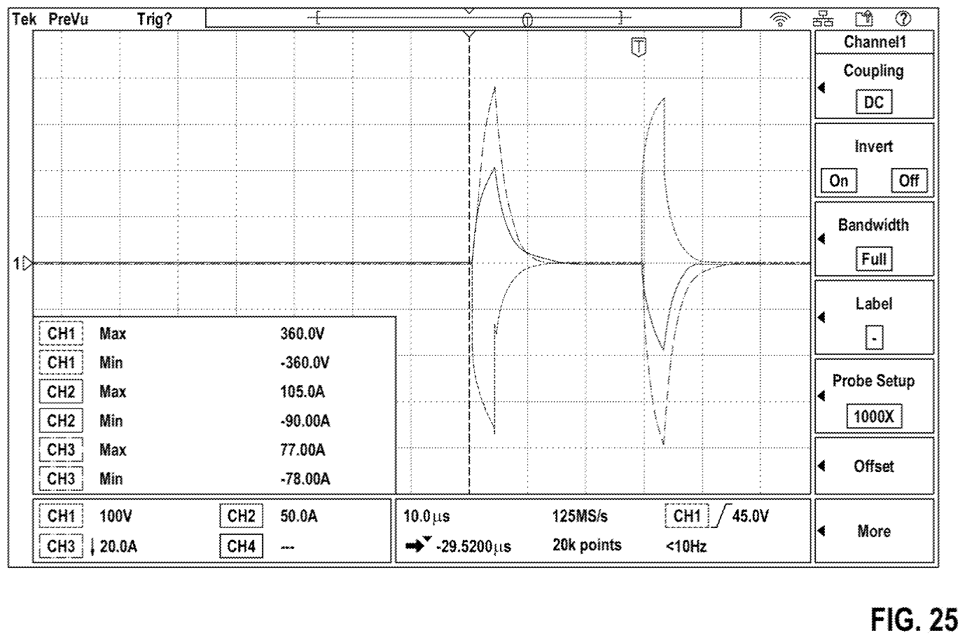

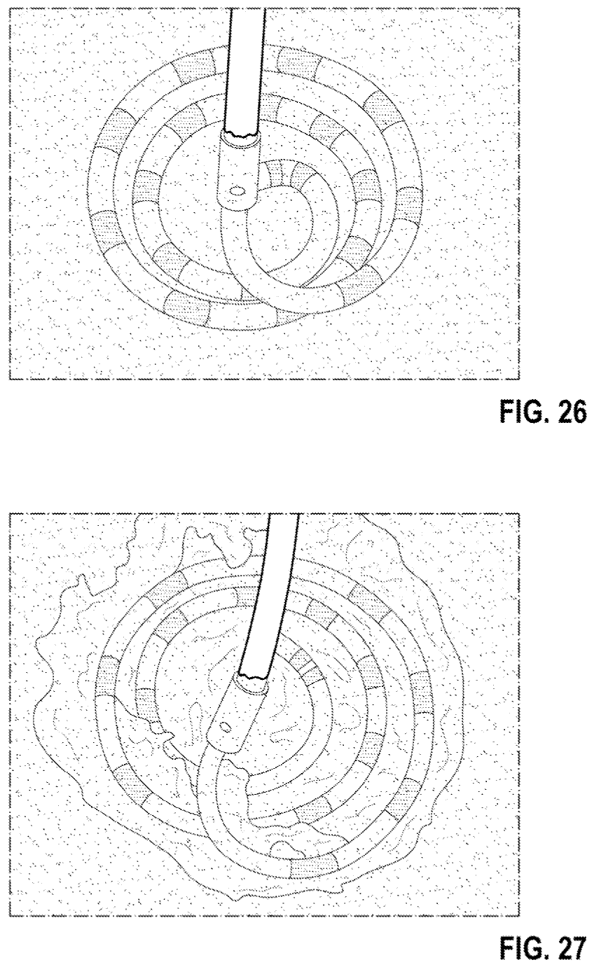

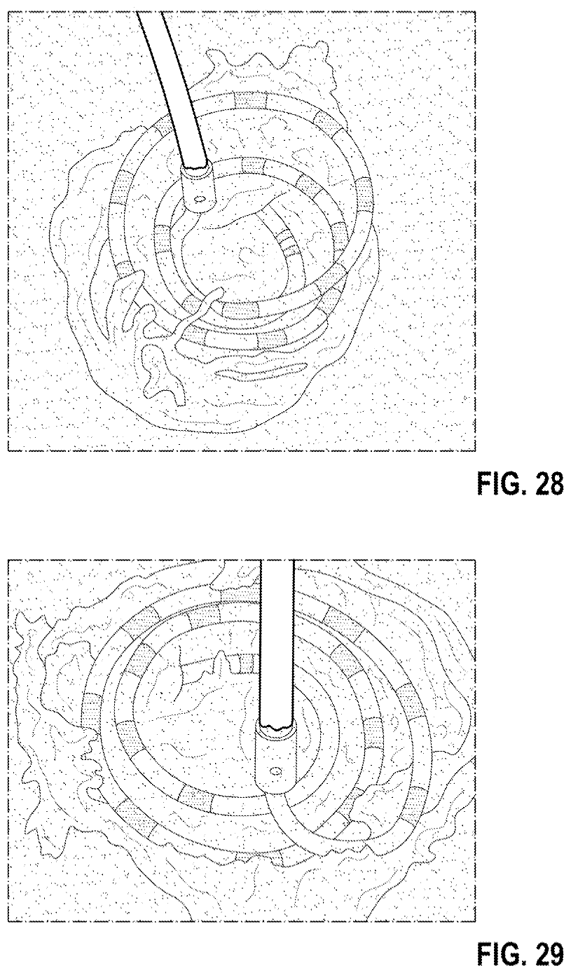

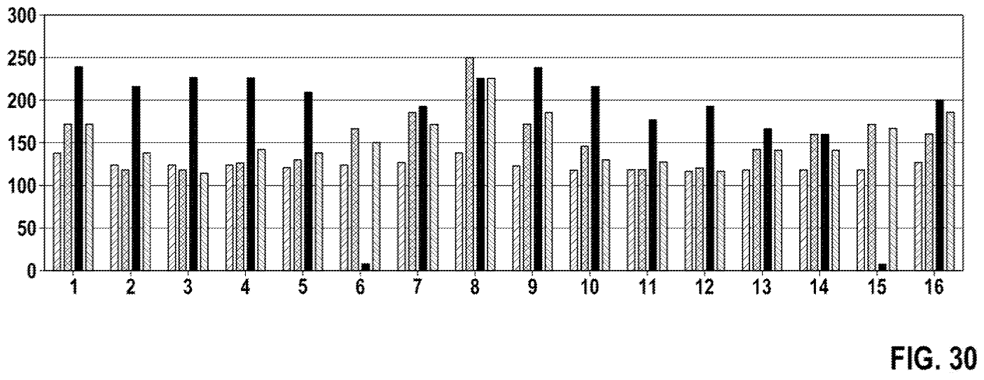

FIGS. 23 and 24 show the current measurements using a single pulse for each of the electrodes in order to determine CU, namely a rectangular pulse. FIG. 23 represents a rectangular current waveform as response to the rectangular voltage pulse. The tooth shaped waveform shown in FIG. 24 represents the measured current in the case of a short circuit. Even in this case a current measurement and thereby impedance measurement is possible. Current measurements (all even electrodes, 16 single electrodes) have been performed with a current transformer (Magnelab CT-C0.5) while a 500 V rectangular biphasic pulse (4 μs pulse length, 25 μs interphase delay) was applied. The impedances determined from the peak current measurement values are displayed as bars for each electrode (electrode number at x-axis) and impedance (in Ω at y-axis). The first (dark blue) bars refer to the position shown in FIG. 26 (ablation portion in saline), the second (orange) bars refer to the position shown in FIG. 27, the third (grey) bars refer to the position shown in FIG. 28, and the fourth (yellow) bars refer to the position shown in FIG. 29.

The impedance values shown for the saline configuration are low because of the higher conductivity of saline (˜0.7 S/m, which is matched to human blood in this experiment) compared to the chicken heart tissue. For the position shown in FIG. 27 the electrodes 2 to 5 and 11 to 13 have lesser contact, whereas the other electrodes have better contact. Regarding the position shown in FIG. 28 the electrodes 6 and 15 are short circuited and the position of the ablation portion needs to be corrected (impedance close to zero). The position shown in FIG. 29 provides impedance values similar to the position of FIG. 27.

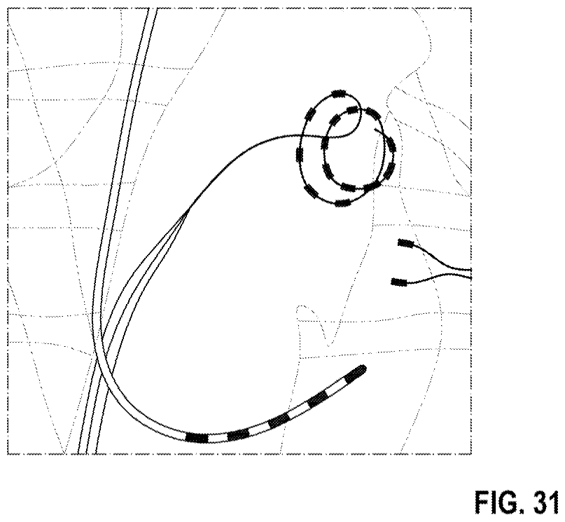

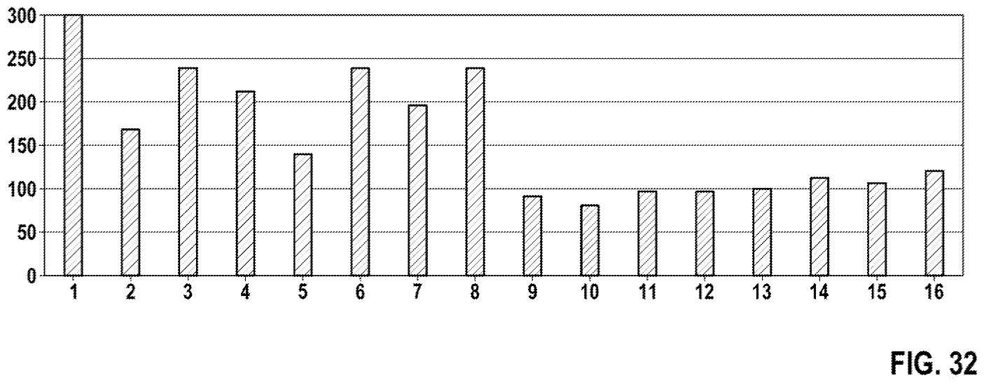

FIG. 31 shows an animal setup. For this test a corkscrew-type catheter (25 mm outer diameter) with 16 electrodes with a spacing of 6 mm (first group of 8 electrodes) and 3 mm (second group of 8 electrodes) was positioned at the right ventricular outflow tract of an 80 kg pig. Rectangular pulses with an amplitude of 500 V were used. For calculating impedance, the maximal values of voltage and current were used. FIG. 32 shows the impedance values (in Ω) determined from current measurements as bars in relation to the respective electrode (see x-axis). It can be shown that the electrodes 9 to 16 have a quite good contact uniformity, whereas with regard to the electrodes 1 to 8 the contact uniformity can be considered mediocre. However, the impedance values of the first group of electrodes 1 to 8 is higher as the electrodes are at a greater distance compared to the second group of electrodes. Accordingly, if one calculates the IU value for the electrodes, the two groups of electrodes should be differentiated. If one applies the above formula for IU and IU*, one derives IU=0.84 and IU*=0.58. It can be seen that IU* appears to be too low as it does not take the two groups of electrodes into account.

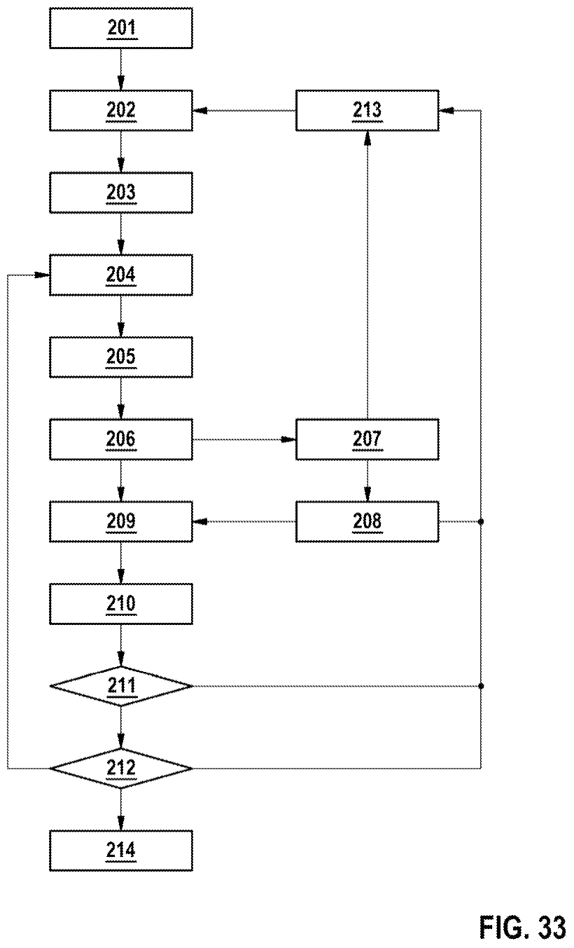

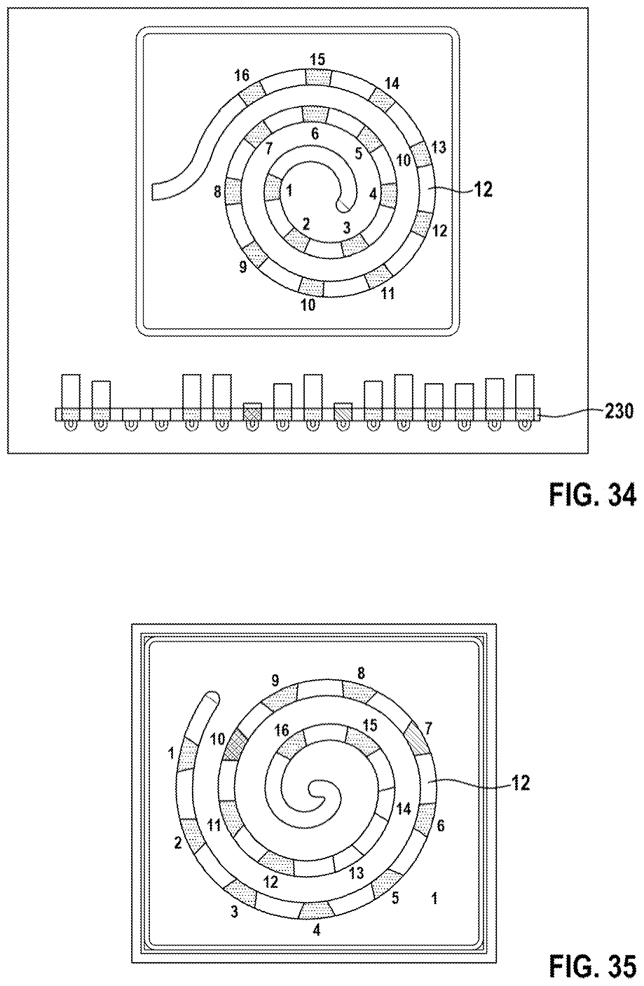

In the following the usage of an inventive catheter as described with regard to FIGS. 1 and 4 is explained in detail referring to the flowchart of FIG. 33. In the first step 201, the catheter 1 is manipulated to targeted PV antrum in the usual way. During advancement of the catheter the ablation portion 12 is covered by the delivery sheath 15 until the distal end of the catheter reaches the targeted region. In the next step 202, the catheter provides quality EGMs to confirm placement near PV and to assess pre-PFA amplitudes and/or an electro-anatomical mapping system displays the 3-dimensional shape and location of the catheter 1. Then, in the next step 203, and after release of the ablation portion 12 from the delivery sheath 15 by retracting the delivery sheath into proximal direction, the AC index and/or CU value measurement is started by short pressing a food pedal of the catheter 1. Then, in step 204, accurate current or impedance measurements between electrodes 120 of the catheter are provided as explained above in detail by the measurement unit 68, the waveform generator 50 and the ECU 70. In one embodiment, the measurement may be provided to all electrodes 120 of the ablation portion 12 or, alternatively, electrodes at positions at risk are measured. Afterwards, the current or impedance measurement values are processed by the ECU 70 and the impedance values for all ablation electrodes, AR indexes of electrode pairs, IU value and/or the CU value for all ablation electrodes of the ablation portion 12 are determined in the following step 205. In step 206, the GUI connected with the ECU 70 colors catheter electrodes or a respective bar diagram at risk of arcing in easy-to-see colors as shown in FIGS. 34 and 35. FIG. 34 depicts the ablation portion 12 with 16 numbered electrodes 120 and a respective bar diagram 230, wherein the height of a bar shown with reference to the electrode number represents the impedance value. The bar diagram shows a low impedance for electrodes number 7 and 10. Electrodes 13 and 14 are mapping electrodes and therefore not measured. FIG. 15 indicates the calculated impedance values directly at the electrode location of electrodes 7 and 10 at the ablation portion 12 with different colors, wherein each color represents the deviation from the target impedance value. The red color of electrode number 10 visualizes a greater deviation from the target impedance value than the yellow color of electrode number 7.

If a risk of arcing is identified and visualized by the GUI (step 207), the electrodes are grouped such that the critical electrodes are split into separate energy-delivery groups (step 208). Now, in step 209, the GUI displays impedances, AR indexes, IU value and/or CU value of electrodes that are in an acceptable range. If there is no risk of arcing identified step 209 can be directly reached from step 206. Then, in step 210, a PFA treatment is initiated by, e.g. a food pedal of the ablation catheter is continued to be pressed (e.g. by some seconds) by the HCP to the patient if an acceptable positioning of the ablation catheter is shown. Then, in step 211, the procedure continues with step 204 if there was no PFA precheck measurement, with step 212 if the PFA precheck measurement is OK, and with step 213 if the PFA precheck measurement failed. Step 213 contains a repositioning of the catheter, in particular of its ablation portion 12 with respect to the targeted PV antrum. After step 213 the procedure continues with step 202 (see above).

Then, if PFA delivery is aborted by the user in step 212, the procedure continues with step 213 (see explanation of step 213 above). If the PFA delivery is not aborted during treatment, the procedure continues with step 214 the PFA generator provides accurate delivery of ablation energy according to pulse protocol to the user by the electrodes 120 of the ablation portion 12.

According to above procedure, the PFA arcing risk and/or contact uniformity is checked prior PFA ablation in order to guarantee the catheter position with the highest contact uniformity and lowest arcing risk for all electrodes taking part in the PFA. Accordingly, dangerous arcing can be avoided and the electrodes have a uniform contact to the targeted tissue in order to provide high-quality PFA realizing a moat of electrical isolation in one shot.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

What is claimed is:

1. A system for treatment of patient tissue by delivery of high-voltage pulses, comprising:
an ablation catheter,
a measurement unit, and
an electronic control unit (ECU),
wherein the catheter comprises a catheter shaft, and an ablation portion being arranged at a distal end of the catheter shaft, with a plurality of electrodes accommodated along the ablation portion, wherein each of the plurality of electrodes is electrically connected to the measurement unit through the catheter shaft,
wherein the measurement unit is configured to perform measurements using an energy source thereby determining measurement values of a subgroup of the plurality of electrodes,
wherein said subgroup is formed by all or a part of the plurality of electrodes,
wherein the ECU is configured to receive and analyze said measurement values provided by the measurement unit and determine arcing risk (AR) and/or a contact uniformity (CU) and/or impedance uniformity (IU) value indexes for said subgroup of the plurality of electrodes.

2. The system of claim 1, wherein said measurement values are bipolar impedance measurement values of electrode pairs of a subgroup of the plurality of electrodes and/or quasi-unipolar impedance measurement values of a subgroup of the plurality of electrodes and/or current measurement values of a subgroup of the plurality of electrodes.

3. The system of claim 2, wherein the impedance and/or current measurement values are determined as response to an alternating voltage and/or at least one voltage pulse.

4. The system of claim 1, wherein the determined arcing risk (AR) and/or a contact uniformity (CU) indexes are based on said impedance measurement values

5. The system of claim 1, wherein the impedance uniformity (IU) indexes are based on said current measurement values.

6. The system of claim 1, wherein the electronic control unit is arranged proximal to or at the proximal end of the catheter, and wherein the measurement unit is connected to or integrated within the ECU

7. The system of claim 1, wherein the measurement unit is configured to determine at least one current measurement value for each of the subgroup of the plurality of electrodes by measuring the respective current value of one or several of rectangular, sinusoidal, tooth or similar shaped voltage pulses, wherein one impedance value is determined from said determined current measurement values for each of the subgroup of electrodes.

8. The system of claim 2, wherein the ECU is configured to determine an impedance uniformity (IU) of two groups of the subgroup of electrodes, wherein
$$IU = 1 - \frac{1}{2} ( \frac{σ ( \{ {Z}_{d} \} )}{μ ( \{ {Z}_{d} \} )} + \frac{σ ( \{ {Z}_{p} \} )}{μ ( \{ {Z}_{p} \} )} )$$
wherein σ({Z_{d,p}}) is the standard deviation and μ({Z_{d,p}}) the mean value of the determined impedances of the electrodes of the respective group.

9. The system of claim 1, wherein the ECU is configured to determine the AR index for a particular electrode pair x,y from the bipolar impedance measurement values of the particular electrode pair x,y from the subgroup of electrodes scaled by the minimum of bipolar impedance measurement values of the respective electrodes with their adjoining electrodes of the subgroup.

10. The system of claim 1, wherein the ECU is configured to determine the CU value for the subgroup of electrodes based on the standard deviation of the bipolar impedance measurement values of the pairs of adjoining electrodes of said subgroup or based on the minimum and the maximum of the bipolar impedance measurement values of the pairs adjoining electrodes of said subgroup and/or to determine the CU value for the subgroup of electrodes based on the standard deviation of a quasi-unipolar impedance measurement values of all electrodes of said subgroup or based on the minimum and the maximum of the quasi-unipolar impedance measurement values of all electrodes of said subgroup.

11. The system of claim 1, wherein the ECU is configured to determine an overall risk for arcing for all electrodes of the subgroup based on a maximum of the AR index of all electrode pairs of the subgroup.

12. The system of claim 1, wherein the measurement unit is configured such that the frequency for determination of quasi-unipolar or bipolar impedance measurement values of the subgroup of electrodes is between 1 kHz and 1 MHz and/or such that the voltage amplitude of the pulses is between 1V and 1 kV, in particular between 10V and 700V, in particular between 100V and 500V

13. A method for assessment of positions and/or configuration of a plurality of electrodes of an ablation catheter for treatment of patient tissue by delivery of high-voltage pulses comprising a catheter shaft and an ablation portion, wherein the ablation portion is arranged at a distal end of the catheter shaft with the plurality of electrodes accommodated along the ablation portion, wherein each of the plurality of electrodes is electrically connected to a measurement unit through the catheter shaft, wherein the measurement unit performs measurements using an energy source thereby determining measurement values of a subgroup of the plurality of electrodes, wherein the subgroup is formed by all electrodes or a part of the plurality of electrodes, respectively, wherein said measurement values are transmitted to the ECU which receives and analyzes said measurement values as well as determines arcing risk (AR) and/or a contact uniformity (CU) and/or an impedance uniformity (IU) indexes based on said current measurement values.

14. The method of claim 13, wherein said measurement values are bipolar impedance measurement values of electrode pairs of a subgroup of the plurality of electrodes and/or quasi-unipolar impedance measurement values of a subgroup of the plurality of electrodes and/or current measurement values of a subgroup of the plurality of electrodes.

15. The method of claim 14, wherein the impedance and/or current measurement values are determined as response to an alternating voltage and/or at least one voltage pulse.

16. The method of claim 13, wherein the determined arcing risk (AR) and/or a contact uniformity (CU) indexes are based on said impedance measurement values

17. The method of claim 13, wherein the impedance uniformity (IU) indexes are based on said current measurement values.

18. The method of claim 13, wherein the electronic control unit is arranged proximal to or at the proximal end of the catheter, and wherein the measurement unit is connected to or integrated within the ECU

19. The method of claim 13, wherein the electronic control unit is arranged separate from catheter, and wherein the measurement unit is connected to or integrated within the ECU

20. The method of claim 13, wherein the measurement unit determines at least one current measurement value for each of the subgroup of the plurality of electrodes by measuring the respective current value of one or several of rectangular, sinusoidal, tooth or similar shaped voltage pulses, wherein one impedance value is determined from said determined current measurement values for each electrode of the subgroup of electrodes.

21. The method of claim 20, wherein the ECU determines an impedance uniformity (IU) of two groups of the subgroup of electrodes, wherein
$$IU = 1 - \frac{1}{2} ( \frac{σ ( \{ {Z}_{d} \} )}{μ ( \{ {Z}_{d} \} )} + \frac{σ ( \{ {Z}_{p} \} )}{μ ( \{ {Z}_{p} \} )} ) \vert ,$$
wherein ({Z_{d,p}}) is the standard deviation and μ({Z_{d,p}}) the mean value of the determined impedances of the electrodes of the respective group.

22. The method of claim 13, wherein the ECU determines the AR index for a particular electrode pair x,y from the bipolar impedance measurement values of the particular electrode pair x,y from the subgroup of electrodes scaled by the minimum of bipolar impedance measurement values of the respective electrodes with their adjoining electrodes of the subgroup.

23. The method of claim 13, wherein the ECU determines the CU value for the subgroup of electrodes based on the standard deviation of the bipolar impedance measurement values of the pairs of adjoining electrodes of said subgroup or based on the minimum and the maximum of the bipolar impedance measurement values of the pairs adjoining electrodes of said subgroup and/or determines the CU value for the subgroup of electrodes based on the standard deviation of the quasi-unipolar impedance measurement values of all electrodes of said subgroup or based on the minimum and the maximum of the quasi-unipolar impedance measurement values of all electrodes of said subgroup.

24. The method of claim 13, wherein ECU determines an overall risk for arcing for all electrodes of the subgroup based on a maximum of the AR index of all electrode pairs of the subgroup.

25. A computer program product comprising instructions which, when executed by a processor, cause the processor to perform the steps of the method according to claim 13.

26. A computer readable data carrier storing a computer program product according to claim 25.
